(19) United States
(12) Patent Application Publication
(10) Pub. No.: US 2008/0119508 A1
(43) Pub. Date: May 22, 2008

(54) Multi-Route Administration Of Immune Response Modifier Compounds

(75) Inventors: Herbert B. Slade, Woodbury, MN (US); Cynthia A. Guy, Osseo, MN (US)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, St. Paul, MN (US)

(21) Appl. No.: 11/722,585

(22) PCT Filed: Dec. 28, 2005
(86) PCT No.: PCT/US05/47069

§ 371 Date: Jun. 22, 2007

Related U.S. Application Data
(60) Provisional application No. 60/640,873, filed on Dec. 30, 2004.

(51) Int. Cl. A61K 31/4745; A61P 35/00; A61P 31/00

(52) U.S. Cl. 514/293

(57) ABSTRACT

A method of treating disease with immune response modifier (IRM) compounds by using at least two different routes of administration, such as administering at least one IRM to a subject locally (e.g., topically) at a disease site in combination with separately administering at least one IRM to the subject systemically (e.g., orally or by injection).

### CROSS-REFERENCE TO RELATED APPLICATIONS

This application claims priority to U.S. provisional application 60/640,873, filed Dec. 30, 2004, the entire contents of which is hereby incorporated by reference.

### FIELD OF THE INVENTION

The present invention relates to administration of immune response modifier (IRM) compounds for use in treating disease.

### BACKGROUND

There has been a major effort in recent years, with substantial progress being made, to develop drugs that can beneficially modify the immune system. For examples, various imidazoquinoline amine, imidazopyridine amine, 6,7-fused cycloalkylimidazopyridine amine, 1,2-bridged imidazoquinoline amine, thiazoloquinoline amine, oxazoloquinoline amine, thiazolopyridine amine, oxazolopyridine amine, imidazonaphthyridine amine, imidazotetrahydronaphthyridine amine, and thiazolonaphthyridine amine compounds have demonstrated potent immunostimulating, antiviral and antitumor (including anticancer) activity, and have also been shown to be useful as vaccine adjuvants and treatment of TH2-mediated diseases.

The mechanism for the immunostimulatory activity of these IRM compounds is thought to be due in substantial part to enhancement of the immune response by induction of various important cytokines (e.g., interferons, interleukins, tumor necrosis factor, etc.). Such compounds have been shown to stimulate a rapid release of certain monocyte/macrophage-derived cytokines and are also capable of stimulating B cells to secrete antibodies, which play an important role in these IRM compounds' activities. One of the predominant immunostimulating responses to these compounds can be the induction of interferon (IFN)-α production, which is believed to be very important in the acute antiviral and antitumor activities seen. Moreover, up regulation of other cytokines such as, for example, tumor necrosis factor (TNF), Interleukin-1 (IL-1), IL-6, and IL-12 also have potentially beneficial activities and are believed to contribute to the antiviral, antitumor, and other properties of these compounds.

One area of particular interest for IRMs has been treatment of skin cancers, the incidence of which has been rapidly increasing worldwide. The drug product Aldara™ (containing the IRM compound imiquimod) has recently been approved for treatment of superficial basal cell carcinoma (BCC), as well as actinic keratosis.

However, surgical removal is still by far the most common treatment for skin cancers, including melanomas, BCCs, and SCCs. This can take the form of electrodesiccation and curettage, cryosurgery, simple wide excision, micrographic surgery, or laser therapy. Other treatments, used when the cancers are detected at a later stage of development, are external radiation therapy, chemotherapy, or to a lesser extent, bio-immunotherapy or photodynamic therapy. Unfortunately, though, there has been very limited success in treating or preventing recurrence of these cancers, especially malignant melanoma, once they have reached more advanced stages. Follow-up surgery is often necessary, with the risk of further disfigurement and scarring. And once the cancer has metastasized there is a high risk of mortality.

Accordingly, there is a continuing need for new treatment methods to provide the enhanced therapeutic benefit from IRM compounds, particularly for conditions such as life-threatening cancers.

### SUMMARY

It is now believed that there is a benefit to treating conditions with IRM compounds where one or more IRM compounds is administered via at least two distinct routes of delivery in combination, for example systemically (e.g., by injection) and locally (e.g., topically). It is believed that this provides a way of synergistically targeting the immune system directly to the disease while boosting the immune response throughout the body, e.g., so as to treat or prevent metastasized cancers or infections that may have spread. In a sense, local administration to a tumor or infection site directly can be used as an opportunity to sensitize the immune system to the specific disease being treated, while the broad immune response induced by non-local application can seek out and target the disease elsewhere in the body.

While applicable for many diseases, it is believed that this new approach will provide benefits in treating malignant melanoma, which despite massive efforts has remained one of the most difficult cancers to treat. Although topical Aldara has shown some activity against melanoma skin lesions, surgery is the conventional treatment. However, it is believed that by administering IRMs locally to the melanoma lesion site on the skin (even after the main lesion has been surgically removed) in combination with systemic delivery, there is a better chance of addressing the high risk that the cancer has already metastasized at the time of initial surgery.

Thus, the present invention is directed to multi-route dosing regimes for administration of one or more IRM compounds. In one embodiment, the present invention provides a method of treating disease with an immune response modifier including administering at least one IRM to a subject topically in combination with separately administering at least one IRM to the subject systemically.

Such multi-route regimens are useful for treating a variety of diseases including cancer (e.g., melanoma and carcinomas) as well as viral, fungal, protazoal, or bacterial infections. Such multi-route regimens are particularly useful for treating melanoma, in particular, by applying at least one IRM topically (or locally via, e.g., subcutaneous, intra-dermal, or intra-tumoral injection) to a melanoma lesion and separately administering at least one IRM systemically.

Herein, topical application involves application to dermal and mucosal tissues, including vaginal, rectal, nasal, buccal, and pulmonary applications. Herein, systemic application involves oral and parenteral (including subcutaneous (subQ or SC) if the intended result is systemic distribution as opposed to local delivery into a lesion), intramuscular (IM), intraperitoneal (IP), intravenous (IV), intrathecal, intraventricular, etc.) administration.

The IRM can be selected from the group consisting of imidazoquinoline amines, tetrahydroimidazoquinoline amines, imidazopyridine amines, 6,7-fused cycloalkylimidazopyridine amines, 1,2-bridged imidazoquinoline amines, imidazonaphthyridine amines, imidazotetrahydronaphthyridine amines, oxazoloquinoline amines, thiazoloquinoline amines, oxazolopyridine amines, thiazolopyridine amines, oxazolonaphthyridine amines, thiazolonaphthyridine amines, 1H-imidazo dimers fused to pyridine amines, quinoline amines, tetrahydroquinoline amines, naphthyridine amines, tetrahydronaphthyridine amines, and combinations thereof.

The present invention also provides an aqueous pharmaceutical composition suitable for parental administration. The composition includes: an immune response modifier compound (IRM); a pharmaceutically acceptable acid (e.g., citric acid, hydrochloric acid, lactic acid, acetic acid, or aspartic acid); a tonicity adjuster (e.g., mannitol, glycerin, sorbitol, or dextrose); sterile water; and optionally a pH adjuster (e.g., NaOH); with the proviso that the IRM is other than 1-(2-methylpropyl)-1H-imidazo[4,5-c]quinolin-4-amine or 4-amino-α,α-dimethyl-1H-imidazo[4,5-c]quinoline-1-ethanol.

The invention includes, but is not limited to, the following embodiments:
1. A method of treating disease with immune response modifiers (IRMs) by administering at least one IRM compound via at least two different routes of delivery.
2. The method of embodiment 1, wherein there is only one IRM compound active moiety is used.
3. The method of embodiment 2, wherein two different salt forms of the IRM compound active moiety are used.
4. The method of embodiment 1, wherein at least two different IRM compound active moieties are used.
5. The method of any preceding embodiment, wherein the routes of delivery include local delivery and systemic delivery.
6. The method in claim 5, wherein the local route of delivery is topical delivery.
7. The method of embodiment 6, wherein topical delivery is achieved using an IRM-containing gel or cream formulation.
8. The method of embodiments 5, 6 or 7, wherein systemic delivery is achieved by injection or oral delivery.
9. The method of any preceding embodiment, wherein the disease being treated is cancer.
10. The method of embodiment 9, wherein an IRM is delivered locally directly to the cancer and an IRM is delivered systemically to the entire body.
11. The method of embodiment 10, wherein the IRM delivered locally is injected directly into the cancer.
12. The method of any one of embodiments 1 through 8, wherein the disease is a viral, fungal, protazoal, or bacterial infection.
13. A method of treating melanoma with an immune response modifier (IRM), the method comprising:
   applying at least one IRM topically to a melanoma lesion on a subject in combination with separately administering at least one IRM to the subject systemically.
14. The method of embodiment 13, wherein the IRM administered topically is administered to a dermal or mucosal tissue.
15. The method of embodiment 14 wherein the IRM administered topically is administered to a vaginal, rectal, nasal, buccal, or pulmonary surface.
16. The method of any preceding embodiment wherein the IRM is a compound having a 2-aminopyridine fused to a five membered nitrogen-containing heterocyclic ring.
17. The method of embodiment 16 wherein the immune response modifier is selected from the group consisting of imidazoquinoline amines, tetrahydroimidazoquinoline amines, imidazopyridine amines, 6,7-fused cycloalkylimidazopyridine amines, 1,2-bridged imidazoquinoline amines, imidazonaphthyridine amines, imidazotetrahydronaphthyridine amines, oxazoloquinoline amines, thiazoloquinoline amines, oxazolopyridine amines, thiazolopyridine amines, oxazolonaphthyridine amines, thiazolonaphthyridine amines, 1H-imidazo dimers fused to pyridine amines, quinoline amines, tetrahydroquinoline amines, naphthyridine amines, or tetrahydronaphthyridine amines, and combinations thereof.
18. The method of embodiment 16, wherein the immune response modifier is selected from the group consisting of imidazoquinoline amines, tetrahydroimidazoquinoline amines, imidazopyridine amines, and combinations thereof.
19. The method of embodiment 16, wherein the immune response modifier is selected from the group consisting of amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl or heteroaryl substituted imidazoquinoline amines, amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, thioether substituted imidazopyridine amines, and combinations thereof.
20. The method of embodiment 19, wherein the immune response modifier is selected from the group consisting of amide substituted imidazoquinoline amines, sulfonamide substituted imidazoquinoline amines, urea substituted imidazoquinoline amines, thioether substituted imidazoquinoline amines, 7-aryl substituted imidazoquinoline amines, 7-heteroaryl substituted imidazoquinoline amines, sulfonamide substituted tetrahydroimidazoquinoline amines, and combinations thereof.
21. The method of embodiment 17, wherein the immune response modifier is an imidazoquinoline amine.
22. The method of embodiment 19, wherein the immune response modifier is a sulfonamide substituted imidazoquinoline amine.
23. The method of embodiment 16, wherein the immune response modifier is selected from the group consisting of N-[4-(4-amino-2-ethyl-1H-imidazo[4,5-c]quinolin-1-yl)butyl]methanesulfonamide, N-{2-[4-amino-2-(ethoxymethyl)-1H-imidazo[4,5-c]quinolin-1-yl)butyl]-1,1-dimethyleethyl}methanesulfonamide, pharmaceutically acceptable salts thereof, and combinations thereof.
24. The method of any preceding embodiment, wherein an IRM is administered systemically in a formulation comprising:
   a pharmaceutically acceptable acid;
   a tonicity adjuster;
   sterile water; and
   optionally a pH adjuster;
   with the proviso that the IRM is other than 1-(2-methylpropyl)-1H-imidazo[4,5-c]quinolin-4-amine or 4-amino-α,α-dimethyl-1H-imidazo[4,5-c]quinoline-1-ethanol.
25. The method of embodiment 24, wherein the formulation comprises 0.4 wt-% to 0.5 wt-% citric acid, 4 wt-% to 5 wt-% mannitol, and water, wherein the formulation is adjusted to a pH of 5 with the pH adjuster.
26. An aqueous pharmaceutical composition suitable for parental administration comprising:
   an immune response modifier compound (IRM);
   a pharmaceutically acceptable acid;
   a tonicity adjuster;
   sterile water; and optionally a pH adjuster;
   with the proviso that the IRM is other than 1-(2-methylpropyl)-1H-imidazo[4,5-c]quinolin-4-amine or 4-amino-α,α-dimethyl-1H-imidazo[4,5-c]quinoline-1-ethanol.

The terms “comprises” and variations thereof do not have a limiting meaning where these terms appear in the description and embodiments.

As used herein, “a,” “an,” “the,” “at least one,” and “one or more” are used interchangeably.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present invention provides a multi-route dosing regime for administration of one or more IRM compounds. In one embodiment, the present invention provides a method of treating disease with an immune response modifier including administering at least one IRM to a subject locally (e.g., topically or via injection into a lesion) in combination with separately administering at least one IRM to the subject systemically. Herein, the subject is typically a mammal, and may be a human.

The IRM administered by one route (e.g., topically) may be the same or different than the IRM administered via another route (e.g., systemically). Alternatively, the IRM may be the same compound, or may be the same compound active moiety but in a different salt form thereof.

The multi-route regimens of the present invention are useful for treating a variety of diseases including cancer (e.g., melanoma and carcinomas) as well as viral, fungal, protazoal, or bacterial infections. Herein, treating includes therapeutic and/or prophylactic treating.

Such multi-route regimens are particularly useful for treating melanoma, although other types of cancers can be treated. For example, in one approach, an IRM is injected directly into a tumor for local effect and especially to target the immune system to the tumor, and an IRM is also delivered systemically to boost body-wide immune response to the tumor (especially if there is a risk it has metastasized). Non-limiting examples of other cancers for which the present invention may be useful include breast cancer, stomach cancer, colorectal cancer, prostate cancer, testicular cancer, head and neck cancer, lung cancer, etc. Any cancer where there is a localized tumor site to which an IRM can be delivered, in combination with administration via a different route of delivery (which will often be systemic delivery, but could be other routes as well).

In some embodiments, IRMs of the present invention may be administered to the subject in combination with other modes of treatment. This is particularly true for cancer therapy. Such other modes of cancer therapy include, but are not limited to, radiation treatment, brachytherapy, external beam radiation, chemotheraphy, hormone therapy, immunomodulatory therapy, therapeutic vaccine therapy, and antibody therapy. The administration of the agents of the present invention can take place before, during, or after the other therapy. Likewise, the IRMs may be delivered via different routes simultaneously or at different times.

For cancer treatment, the efficacy of treatment may be assessed by various parameters well known in the art. This includes, but is not limited to, determination of tumor size, location and vascularization, as determined by such methods including, but not limited to, X-rays, scans, magnetic resonance imaging, computerized tomography, and/or various nuclear medicine techniques and algorithms to evaluate tumor size and burden in three dimensions. Angiography can be used to evaluate vascularization of tumors and other tissues. Other methods of determining tumor location, stage, and grade include, but are not limited to, gene arrays, immuno-histochemistry, and/or other techniques for measuring biomarkers relevant to assessing a disease.

The efficacy of the administration of an IRM effective for the treatment of cancer may be demonstrated by such means, including, but not limited to, the inhibition of tumor growth, the inhibition of tumor progression, the inhibition of tumor spread, the inhibition of tumor invasiveness, the inhibition of tumor vascularization, the inhibition of tumor angiogenesis, and/or the inhibition of tumor metastasis.

The inhibition of tumor growth is a decrease in the growth rate of a tumor. It includes, but is not limited to, at least one of a decrease in tumor weight or tumor volume, a decrease in tumor doubling time, a decrease in the growth fraction or number of tumor cells that are replicating, a decrease in the rate in which tumor cells are shed, and/or a decrease in the ratio of cell production to cell loss within a tumor. The inhibition of tumor growth can also include the inhibition of tumor growth of primary lesions and/or any metastatic lesions.

For oral cancer, the inhibition of tumor progression includes the disruption or halting of the progression of premalignant lesions, also called leukoplakia, to malignant carcinoma.

The inhibition of tumor spread is the decrease in the dissemination of a tumor to other locations. This dissemination to other locations can be the result of the seeding of a body cavity or surface with cancerous cells from a tumor and/or the transport of tumor cells through the lymphatic system and/or circulatory system. The inhibition of tumor spread can also include the inhibition of tumor spread in primary lesions and/or any metastatic lesions.

The inhibition of tumor invasiveness is the decrease in the infiltration, invasion, and/or destruction of the surrounding local tissues, including, but not limited to organs, blood vessels, lymphatics, and/or body cavities. The inhibition of tumor invasiveness can also include the inhibition of tumor invasiveness in primary lesions and/or any metastatic lesions.

The inhibition of tumor vascularization is the decrease in the formation of blood vessels and lymphatic vessels within a tumor and to and from a tumor. The inhibition of tumor vascularization can also include the inhibition of tumor vascularization in primary lesions and/or any metastatic lesions.

The inhibition of tumor angiogenesis is a decrease in the formation of new capillaries and microvessels within a tumor. The inhibition of tumor angiogenesis can also include the inhibition of tumor angiogenesis in primary lesions and/or any metastatic lesions.

The inhibition of tumor metastasis is a decrease in the formation of tumor lesions that are discontinuous with the primary tumor. With metastasis, tumor cells break loose from the primary lesion, enter blood vessels or lymphatics and produce a secondary growth at a distant site. In some cases the distribution of the metastases may be the result of the natural pathways of the drainage of the lymphatic and/or circulatory system. In other cases, the distribution of metastases may be the result of a tropism of the tumor to a specific tissue or organ. For example, prostate tumors may preferentially metastasis to the bone. The tumor cells of a metastatic lesion may in turn metastasize to additional locations. This may be referred to as a metastatic cascade. Tumor cells may metastasize to sites including, but not limited to, liver, bone, lung, lymph node, spleen, brain or other nervous tissue, bone marrow, or an organ other than the original tissue of origin. The inhibition of tumor metastasis includes the inhibition of tumor metastasis in primary lesions and/or any metastatic lesions.

Herein, local application includes, e.g., topical application as well as injectable applications (e.g., intra-dermal, intra-tumoral, or subcutaneous) intended for local distribution only, without substantial systemic delivery. Herein, topical application involves application to dermal and mucosal tissues, including vaginal, rectal, nasal, buccal, and pulmonary applications. Herein, systemic application involves oral and parenteral (including subcutaneous (subQ or SC) if intended for systemic distribution as opposed to local administration), intramuscular (IM), intraperitoneal (IP), intravenous (IV), intrathecal, intraventricular, etc.) administration. Thus, formulations of the present invention can be administered to a subject (e.g., mammal, particularly a human) in various ways, for example, by spraying, injection, inhalation (e.g., from a nebulizer or spray pump atomizer), gel, cream, foam, transdermal patch, suppository, etc.

Formulations of the present invention suitable for topical administration are disclosed in, e.g., U.S. Patent Publication No. US 2003/0199538 and International Publication No. WO 2003/045391. A typical formulation for topical administration includes, for example, isostearic acid (e.g., 15-35 wt-%), medium-chain triglycerides (e.g., 5-10 wt-%), propylene glycol (e.g., 5-10 wt-%), parabens (e.g., methyl, ethyl, and mixtures thereof) (e.g., 0.1-0.5 wt-%), edetate disodium (e.g., 0.01-0.1 wt-%), polymers such as CARBOMERS and POLOXAMERS (e.g., 4.0-5.0 wt-%), and water (preferably sterile water), wherein the formulation is optionally adjusted to a desired pH, preferably a pH of 5.8 (e.g., by NaOH). An IRM can be incorporated into such a formulation in a variety of concentrations.

Formulations of the present invention suitable for parenteral administration conveniently include a sterile aqueous preparation of the desired compound, or dispersions of sterile powders including the desired compound, which are preferably isotonic with the blood of the subject. Isotonic agents that can be included in the liquid preparation include sugars, buffers, and salts such as sodium chloride. Solutions of the desired compound can be prepared in water, optionally mixed with a nontoxic surfactant. Dispersions of the desired compound can be prepared in water, ethanol, a polyol (such as glycerol, propylene glycol, liquid polyethylene glycols, and the like), vegetable oils, glycerol esters, and mixtures thereof.

A typical formulation for systemic administration (e.g., IV and SC) includes, for example, citric acid or other pharmaceutically acceptable acid (e.g., hydrochloric acid, lactic acid, acetic acid, aspartic acid), mannitol or other tonicity adjuster (e.g., glycerin, sorbitol, dextrose), and water (preferably, sterile water), wherein the formulation is optionally adjusted to a desired pH, preferably a pH of 5, by a suitable pH adjuster (e.g., by NaOH). The pharmaceutically acceptable acid is preferably present in the formulation (i.e., composition) in an amount of at least 0.4 wt-%, although lower concentrations, such as 0.3%, may also be used, and preferably no more than 0.5 wt-%, based on the total weight of the formulation. The tonicity adjuster is preferably present in the formulation in an amount of at least 4 wt-%, and preferably no more than 5 wt-%, based on the total weight of the formulation. Additional information regarding formulations for injection can be found in co-pending application attorney docket number 61658WO003, entitled Immune Response Modifier Formulations and Methods, filed even date herewith.

The IRM can be incorporated into such a formulation in a variety of concentrations. Typical formulations include one or more IRMs in amounts of at least 0.001 wt-%, and preferably at least 0.2 wt-%, and even up to 1.5 wt-%, based on the total weight of the formulation. An IRM can be incorporated into such a formulation in a variety of concentrations.

Formulations of the present invention suitable for oral administration can include those discussed above for systemic administration, wherein the formulations are suitably diluted. For example, such formulations can be diluted with dextrose or other suitable diluents to a total volume of 10 mL.

Other oral formulations may include discrete units such as tablets, troches, capsules, lozenges, wafers, or cachets, each containing a predetermined amount of the IRM, as a powder, in granular form, incorporated within liposomes, or as a solution or suspension in an aqueous liquid or non-aqueous liquid such as a syrup, an elixir, an emulsion, or a draught.

The tablets, troches, pills, capsules, and the like may also contain one or more of the following: a binder such as gum tragacanth, acacia, corn starch, or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The compound may be incorporated into sustained-release preparations and devices if desired.

Formulations for rectal or vaginal administration may be presented as a suppository with a suitable carrier such as cocoa butter, or hydrogenated fats or hydrogenated fatty carboxylic acids.

Nasal spray formulations can include purified aqueous solutions of the desired compound with preservative agents and isotonic agents. Such formulations are preferably adjusted to a pH and isotonic state compatible with the nasal mucous membranes. Preferably, such formulations are in solution form at room temperature (i.e., 25° C.-30° C.). Also, such formulations are sufficiently low in viscosity (less than 100 centipoise (cps)) at room temperature. At such low viscosity level, the compositions are typically and preferably sprayable. In this context, “sprayable” means the formulation can be delivered using a conventional pump spray device.

The amount of an IRM compound that will be therapeutically effective in a specific situation will depend on such things as the activity of the particular compound, the dosing regimen, the application site, the particular formulation, and the condition being treated. As such, specific administration amounts described herein are only exemplary. Those skilled in the art will be able to determine appropriate therapeutically effective amounts based on the guidance provided herein, information available in the art pertaining to those compounds, and routine testing.

Typical topical formulations include one or more IRMs in amounts of at least 0.01 wt-%, and even up to 3.0 wt-%, based on the total weight of the formulation. A preferred systemic formulation includes one or more IRMs in amounts of 0.1 wt-% to 1.6 wt-%, based on the total weight of the formulation. A typical injection volume of 1.5 mL.

In some embodiments, the methods of the present invention include systemically administering sufficient formulation to provide a dose of IRM compound of, for example, from 10 ng/kg to 50 mg/kg to the subject, although in some embodiments the methods may be performed by administering IRM compound in concentrations outside this range. In some of these embodiments, the method includes systemically administering sufficient formulation to provide a dose of IRM compound of from 100 ng/kg to 5 mg/kg to the subject, for example, a dose of from 1 μg/kg to 1 mg/kg.

In some embodiments, the methods of the present invention include topically administering sufficient formulation of IRM compound, for example, from 0.0001 wt-% to 10 wt-% to the subject, although in some embodiments the methods may be performed by administering IRM compound in concentrations outside this range. In some of these embodiments, the method includes topically administering sufficient formulation of IRM compound from 0.001 wt-% to 5 wt-% to the subject, for example, from 0.01 wt-% to 3 wt-%.

### IRM Compounds

IRM compounds used herein are generally agonists of toll-like receptors (TLRs) 7, 8, and/or 9. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304. Other IRMs include biological molecules such as aminoalkyl glucosaminide phosphates (AGPs) and are described, for example, in U.S. Pat. Nos. 6,113,918; 6,303,347; 6,525,028; and 6,649,172. CpGs and other biological IRMs are considered relatively large molecules and many are TLR 9 agonists.

However, TLR 7 and/or 8 agonists may be preferred, and small-molecule IRMs are generally preferred for methods involving multi-route administration including topical delivery. Examples of small organic molecule IRMs (e.g., molecular weight under about 1000 Daltons, preferably under about 500 Daltons, as opposed to large biologic protein, peptides, and the like) are disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,545,016; 6,545,017; 6,558,951; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; European Patent 0 394 026; U.S. Patent Publication Nos. 2002/0016332; 2002/0055517; 2002/0110840; 2003/0133913; 2003/0199538; and 2004/0014779; and International Patent Publication No. WO 04/058759.

IRM compounds suitable for use in the invention preferably include small-molecule IRM compounds having a 2-aminopyridine fused to a five membered nitrogen-containing heterocyclic ring. Such compounds include, for example, imidazoquinoline amines, including but not limited to, substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, and 6-, 7-, 8-, or 9-aryl or heteroaryl substituted imidazoquinoline amines; tetrahydroimidazoquinoline amines, including but not limited to, amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, and thioether substituted tetrahydroimidazoquinoline amines; imidazopyridine amines, including but not limited to, amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; imidazotetrahydronaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines. Various combinations of these IRMs can be used if desired.

In certain embodiments, the immune response modifier is selected from the group consisting of imidazoquinoline amines, tetrahydroimidazoquinoline amines, imidazopyridine amines, and combinations thereof.

In certain embodiments, the immune response modifier is selected from the group consisting of amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl or heteroaryl substituted imidazoquinoline amines, amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, thioether substituted imidazopyridine amines, and combinations thereof.

In certain embodiments, the immune response modifier is selected from the group consisting of amide substituted imidazoquinoline amines, sulfonamide substituted imidazoquinoline amines, urea substituted imidazoquinoline amines, thioether substituted imidazoquinoline amines, 7-aryl substituted imidazoquinoline amines, 7-heteroaryl substituted imidazoquinoline amines, sulfonamide substituted tetrahydroimidazoquinoline amines, and combinations thereof.

In certain embodiments, the immune response modifier is an imidazoquinoline amine. In certain embodiments, the immune response modifier is a sulfonamide substituted imidazoquinoline amine.

In certain embodiments, the immune response modifier is selected from the group consisting of N-[4-(4-amino-2-ethyl-1H-imidazo[4,5-c]quinolin-1-yl)butyl]methanesulfonamide, N-{2-[4-amino-2-(ethoxymethyl)-1H-imidazo[4,5-c]quinolin-1-yl)butyl]-1,1-dimethyleethyl}methanesulfonamide, 4-amino-α,α-dimethyl-2-ethoxymethyl-1H-imidazo[4,5-c]quinolin-1-ethanol, pharmaceutically acceptable salts thereof, and combinations thereof.

The IRM compounds and salts thereof described herein include any of their pharmaceutically acceptable forms, such as isomers (e.g., diastereomers and enantiomers), solvates, polymorphs, and the like. In particular, if a compound is optically active, the invention specifically includes the use of each of the compound's enantiomers as well as racemic combinations of the enantiomers.

The immune response modifier can, for example, be a salt of an acid selected from the group consisting of a carboxylic acid, a halo acid, sulfuric acid, phosphoric acid, dicarboxylic acid, tricarboxylic acid, and combinations thereof. In certain embodiments, the salt of the immune response modifier can be a salt of an acid selected from the group consisting of hydrobromic acid, hydrochloric acid, lactic acid, glutamic acid, gluconic acid, tartaric acid, succinic acid, and combinations thereof.

### Exemplary IRM Compounds

In certain embodiments of the present invention the IRM compound can be chosen from 1H-imidazo[4,5-c]quinolin-4-amines defined by one of Formulas I-V below:

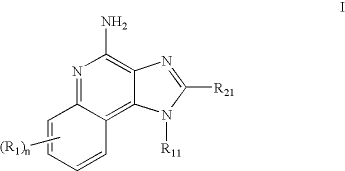

wherein

R₁₁ is selected from alkyl of one to ten carbon atoms, hydroxyalkyl of one to six carbon atoms, acyloxyalkyl wherein the acyloxy moiety is alkanoyloxy of two to four carbon atoms or benzoyloxy, and the alkyl moiety contains one to six carbon atoms, benzyl, (phenyl)ethyl and phenyl, said benzyl, (phenyl)ethyl or phenyl substituent being optionally substituted on the benzene ring by one or two moieties independently selected from alkyl of one to four carbon atoms, alkoxy of one to four carbon atoms and halogen, with the proviso that if said benzene ring is substituted by two of said moieties, then said moieties together contain no more than six carbon atoms;

R₂₁ is selected from hydrogen, alkyl of one to eight carbon atoms, benzyl, (phenyl)ethyl and phenyl, the benzyl, (phenyl)ethyl or phenyl substituent being optionally substituted on the benzene ring by one or two moieties independently selected from alkyl of one to four carbon atoms, alkoxy of one to four carbon atoms and halogen, with the proviso that when the benzene ring is substituted by two of said moieties, then the moieties together contain no more than six carbon atoms; and

each R₁ is independently selected from alkoxy of one to four carbon atoms, halogen, and alkyl of one to four carbon atoms, and n is an integer from 0 to 2, with the proviso that if n is 2, then said R₁, groups together contain no more than six carbon atoms;

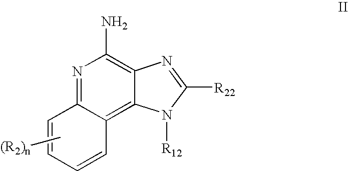

wherein

R₁₂ is selected from straight chain or branched chain alkenyl containing two to ten carbon atoms and substituted straight chain or branched chain alkenyl containing two to ten carbon atoms, wherein the substituent is selected from straight chain or branched chain alkyl containing one to four carbon atoms and cycloalkyl containing three to six carbon atoms; and cycloalkyl containing three to six carbon atoms substituted by straight chain or branched chain alkyl containing one to four carbon atoms; and

R₂₂ is selected from hydrogen, straight chain or branched chain alkyl containing one to eight carbon atoms, benzyl, (phenyl)ethyl and phenyl, the benzyl, (phenyl)ethyl or phenyl substituent being optionally substituted on the benzene ring by one or two moieties independently selected from straight chain or branched chain alkyl containing one to four carbon atoms, straight chain or branched chain alkoxy containing one to four carbon atoms, and halogen, with the proviso that when the benzene ring is substituted by two such moieties, then the moieties together contain no more than six carbon atoms; and

each R₂ is independently selected from straight chain or branched chain alkoxy containing one to four carbon atoms, halogen, and straight chain or branched chain alkyl containing one to four carbon atoms, and n is an integer from zero to 2, with the proviso that if n is 2, then said R₂ groups together contain no more than six carbon atoms;

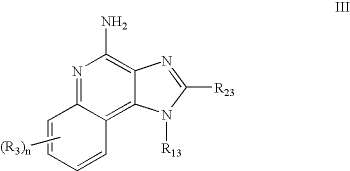

wherein

R₂₃ is selected from hydrogen, straight chain or branched chain alkyl of one to eight carbon atoms, benzyl, (phenyl)ethyl and phenyl, the benzyl, (phenyl)ethyl or phenyl substituent being optionally substituted on the benzene ring by one or two moieties independently selected from straight chain or branched chain alkyl of one to four carbon atoms, straight chain or branched chain alkoxy of one to four carbon atoms, and halogen, with the proviso that when the benzene ring is substituted by two such moieties, then the moieties together contain no more than six carbon atoms; and

each R₃ is independently selected from straight chain or branched chain alkoxy of one to four carbon atoms, halogen, and straight chain or branched chain alkyl of one to four carbon atoms, and n is an integer from zero to 2, with the proviso that if n is 2, then said R₃ groups together contain no more than six carbon atoms;

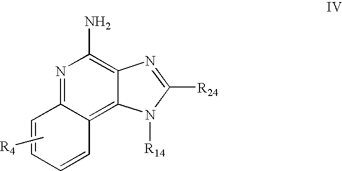

wherein

R₁₄ is —CHRₓR_{y} wherein R_{y} is hydrogen or a carbon-carbon bond, with the proviso that when R_{y} is hydrogen Rₓ is alkoxy of one to four carbon atoms, hydroxyalkoxy of one to four carbon atoms, 1-alkynyl of two to ten carbon atoms, tetrahydropyranyl, alkoxyalkyl wherein the alkoxy moiety contains one to four carbon atoms and the alkyl moiety contains one to four carbon atoms, or 2-, 3-, or 4-pyridyl, and with the further proviso that when R_{y} is a carbon-carbon bond R_{y} and Rₓ together form a tetrahydrofuranyl group optionally substituted with one or more substituents independently selected from hydroxy and hydroxyalkyl of one to four carbon atoms;

R₂₄ is selected from hydrogen, alkyl of one to four carbon atoms, phenyl, and substituted phenyl wherein the substituent is selected from alkyl of one to four carbon atoms, alkoxy of one to four carbon atoms, and halogen; and

R₄ is selected from hydrogen, straight chain or branched chain alkoxy containing one to four carbon atoms, halogen, and straight chain or branched chain alkyl containing one to four carbon atoms;

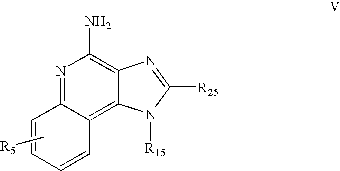

wherein

R₁₅ is selected from hydrogen; straight chain or branched chain alkyl containing one to ten carbon atoms and substituted straight chain or branched chain alkyl containing one to ten carbon atoms, wherein the substituent is selected from cycloalkyl containing three to six carbon atoms and cycloalkyl containing three to six carbon atoms substituted by straight chain or branched chain alkyl containing one to four carbon atoms; straight chain or branched chain alkenyl containing two to ten carbon atoms and substituted straight chain or branched chain alkenyl containing two to ten carbon atoms, wherein the substituent is selected from cycloalkyl containing three to six carbon atoms and cycloalkyl containing three to six carbon atoms substituted by straight chain or branched chain alkyl containing one to four carbon atoms; hydroxyalkyl of one to six carbon atoms; alkoxyalkyl wherein the alkoxy moiety contains one to four carbon atoms and the alkyl moiety contains one to six carbon atoms; acyloxyalkyl wherein the acyloxy moiety is alkanoyloxy of two to four carbon atoms or benzoyloxy, and the alkyl moiety contains one to six carbon atoms; benzyl; (phenyl)ethyl; and phenyl; said benzyl, (phenyl)ethyl or phenyl substituent being optionally substituted on the benzene ring by one or two moieties independently selected from alkyl of one to four carbon atoms, alkoxy of one to four carbon atoms, and halogen, with the proviso that when said benzene ring is substituted by two of said moieties, then the moieties together contain no more than six carbon atoms;

R₂₅ is

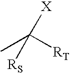

wherein

R_{S} and R_{T} are independently selected from hydrogen, alkyl of one to four carbon atoms, phenyl, and substituted phenyl wherein the substituent is selected from alkyl of one to four carbon atoms, alkoxy of one to four carbon atoms, and halogen;

X is selected from alkoxy containing one to four carbon atoms, alkoxyalkyl wherein the alkoxy moiety contains one to four carbon atoms and the alkyl moiety contains one to four carbon atoms, hydroxyalkyl of one to four carbon atoms, haloalkyl of one to four carbon atoms, alkylamido wherein the alkyl group contains one to four carbon atoms, amino, substituted amino wherein the substituent is alkyl or hydroxyalkyl of one to four carbon atoms, azido, chloro, hydroxy, 1-morpholino, 1-pyrrolidino, alkylthio of one to four carbon atoms; and

R₅ is selected from hydrogen, straight chain or branched chain alkoxy containing one to four carbon atoms, halogen, and straight chain or branched chain alkyl containing one to four carbon atoms;

and pharmaceutically acceptable salts of any of the foregoing.

In another embodiment, the IRM compound can be chosen from 6,7 fused cycloalkylimidazopyridine amines defined by Formula VI below:

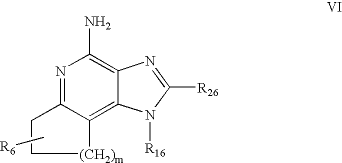

wherein

m is 1, 2, or 3;

R₁₆ is selected from hydrogen; cyclic alkyl of three, four, or five carbon atoms; straight chain or branched chain alkyl containing one to ten carbon atoms and substituted straight chain or branched chain alkyl containing one to ten carbon atoms, wherein the substituent is selected from cycloalkyl containing three to six carbon atoms and cycloalkyl containing three to six carbon atoms substituted by straight chain or branched chain alkyl containing one to four carbon atoms; fluoro- or chloroalkyl containing from one to ten carbon atoms and one or more fluorine or chlorine atoms; straight chain or branched chain alkenyl containing two to ten carbon atoms and substituted straight chain or branched chain alkenyl containing two to ten carbon atoms, wherein the substituent is selected from cycloalkyl containing three to six carbon atoms and cycloalkyl containing three to six carbon atoms substituted by straight chain or branched chain alkyl containing one to four carbon atoms; hydroxyalkyl of one to six carbon atoms; alkoxyalkyl wherein the alkoxy moiety contains one to four carbon atoms and the alkyl moiety contains one to six carbon atoms; acyloxyalkyl wherein the acyloxy moiety is alkanoyloxy of two to four carbon atoms or benzoyloxy, and the alkyl moiety contains one to six carbon atoms, with the proviso that any such alkyl, substituted alkyl, alkenyl, substituted alkenyl, hydroxyalkyl, alkoxyalkyl, or acyloxyalkyl group does not have a fully carbon substituted carbon atom bonded directly to the nitrogen atom; benzyl; (phenyl)ethyl; and phenyl; said benzyl, (phenyl)ethyl or phenyl substituent being optionally substituted on the benzene ring by one or two moieties independently selected from alkyl of one to four carbon atoms, alkoxy of one to four carbon atoms, and halogen, with the proviso that when said benzene ring is substituted by two of said moieties, then the moieties together contain no more than six carbon atoms; and —CHRₓR_{y} wherein

R_{y} is hydrogen or a carbon-carbon bond, with the proviso that when R_{y} is hydrogen Rₓ is alkoxy of one to four carbon atoms, hydroxyalkoxy of one to four carbon atoms, 1-alkynyl of two to ten carbon atoms, tetrahydropyranyl, alkoxyalkyl wherein the alkoxy moiety contains one to four carbon atoms and the alkyl moiety contains one to four carbon atoms, 2-, 3-, or 4-pyridyl, and with the further proviso that when R_{y} is a carbon-carbon bond R_{y} and Rₓ together form a tetrahydrofuranyl group optionally substituted with one or more substituents independently selected from hydroxy and hydroxyalkyl of one to four carbon atoms;

R₂₆ is selected from hydrogen; straight chain or branched chain alkyl containing one to eight carbon atoms; straight chain or branched chain hydroxyalkyl containing one to six carbon atoms; morpholinoalkyl; benzyl; (phenyl)ethyl; and phenyl, the benzyl, (phenyl)ethyl, or phenyl substituent being optionally substituted on the benzene ring by a moiety selected from methyl, methoxy, and halogen; and —C(R_{S})(R_{T})(X) wherein R_{S} and R_{T} are independently selected from hydrogen, alkyl of one to four carbon atoms, phenyl, and substituted phenyl wherein the substituent is selected from alkyl of one to four carbon atoms, alkoxy of one to four carbon atoms, and halogen;

X is selected from alkoxy containing one to four carbon atoms, alkoxyalkyl wherein the alkoxy moiety contains one to four carbon atoms and the alkyl moiety contains one to four carbon atoms, haloalkyl of one to four carbon atoms, alkylamido wherein the alkyl group contains one to four carbon atoms, amino, substituted amino wherein the substituent is alkyl or hydroxyalkyl of one to four carbon atoms, azido, alkylthio of one to four carbon atoms, and morpholinoalkyl wherein the alkyl moiety contains one to four carbon atoms; and

R₆ is selected from hydrogen, fluoro, chloro, straight chain or branched chain alkyl containing one to four carbon atoms, and straight chain or branched chain fluoro- or chloroalkyl containing one to four carbon atoms and at least one fluorine or chlorine atom; and pharmaceutically acceptable salts thereof.

In another embodiment, the IRM compound can be chosen from imidazopyridine amines defined by Formula VII below:

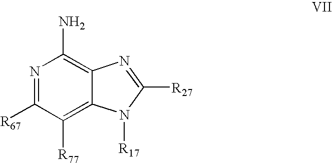

wherein

R₁₇ is selected from hydrogen; —CH₂R_{W} wherein R_{W} is selected from straight chain, branched chain, or cyclic alkyl containing one to ten carbon atoms, straight chain or branched chain alkenyl containing two to ten carbon atoms, straight chain or branched chain hydroxyalkyl containing one to six carbon atoms, alkoxyalkyl wherein the alkoxy moiety contains one to four carbon atoms and the alkyl moiety contains one to six carbon atoms, and phenylethyl; and —CH═CR_{Z}R_{Z} wherein each R_{Z} is independently straight chain, branched chain, or cyclic alkyl of one to six carbon atoms;

R₂₇ is selected from hydrogen; straight chain or branched chain alkyl containing one to eight carbon atoms; straight chain or branched chain hydroxyalkyl containing one to six carbon atoms; alkoxyalkyl wherein the alkoxy moiety contains one to four carbon atoms and the alkyl moiety contains one to six carbon atoms; benzyl, (phenyl)ethyl and phenyl, the benzyl, (phenyl)ethyl and phenyl being optionally substituted on the benzene ring by a moiety selected from methyl, methoxy, and halogen; and morpholinoalkyl wherein the alkyl moiety contains one to four carbon atoms;

R₆₇ and R₇₇ are independently selected from hydrogen and alkyl of one to five carbon atoms, with the proviso that R₆₇ and R₇₇ taken together contain no more than six carbon atoms, and with the further proviso that when R₇₇ is hydrogen then R₆₇ is other than hydrogen and R₂₇ is other than hydrogen or morpholinoalkyl, and with the further proviso that when R₆₇ is hydrogen then R₇₇ and R₂₇ are other than hydrogen;

and pharmaceutically acceptable salts thereof.

In another embodiment, the IRM compound can be chosen from 1,2 bridged imidazoquinoline amines defined by Formula VIII below:

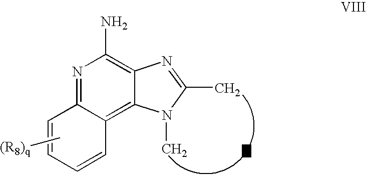

wherein

Z is selected from
—(CH₂)ₚ— wherein p is 1 to 4;
—(CH₂)ₐ—C(R_{D}R_{E})(CH₂)_{b}—, wherein a and b are integers and a+b is 0 to 3, R_{D} is hydrogen or alkyl of one to four carbon atoms, and R_{E} is selected from alkyl of one to four carbon atoms, hydroxy, —ORF wherein R_{F} is alkyl of one to four carbon atoms, and —NR_{G}R′_{G} wherein R_{G} and R′_{G} are independently hydrogen or alkyl of one to four carbon atoms; and
—(CH₂)ₐ—(Y)—(CH₂)_{b}— wherein a and b are integers and a+b is 0 to 3, and Y is O, S, or —NR_{J}— wherein R_{J} is hydrogen or alkyl of one to four carbon atoms;

q is 0 or 1; and

R₈ is selected from alkyl of one to four carbon atoms, alkoxy of one to four carbon atoms, and halogen,

and pharmaceutically acceptable salts thereof.

In another embodiment, the IRM compound can be chosen from thiazoloquinoline amines, oxazoloquinoline amines, thiazolopyridine amines, oxazolopyridine amines, thiazolonaphthyridine amines and oxazolonaphthyridine amines defined by Formula IX below:

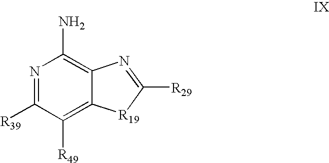

wherein:

R₁₉ is selected from oxygen, sulfur and selenium;

R₂₉ is selected from
-hydrogen;
-alkyl;
-alkyl-OH;
-haloalkyl;
-alkenyl;
-alkyl-X-alkyl;
-alkyl-X-alkenyl;
-alkenyl-X-alkyl;
-alkenyl-X-alkenyl;
-alkyl-N(R₅₉)₂;
-alkyl-N₃;
-alkyl-O—C(O)—N(R₅₉)₂;
-heterocyclyl;
-alkyl-X-heterocyclyl;
-alkenyl-X-heterocyclyl;
-aryl;
-alkyl-X-aryl;
-alkenyl-X-aryl;
-heteroaryl;
-alkyl-X-heteroaryl; and
-alkenyl-X-heteroaryl;

R₃₉ and R₄₉ are each independently:
-hydrogen;
—X-alkyl;
-halo;
-haloalkyl;
—N(R₅₉)₂;
or when taken together, R₃₉ and R₄₉ form a fused aromatic, heteroaromatic, cycloalkyl or heterocyclic ring;

X is selected from —O—, —S—, —NR₅₉—, —C(O)—, —C(O)O—, —OC(O)—, and a bond; and

each R₅₉ is independently H or C₁₋₈alkyl;

and pharmaceutically acceptable salts thereof.

In another embodiment, the IRM compound can be chosen from imidazonaphthyridine amines and imidazotetrahydronaphthyridine amines defined by Formulas X and XI below:

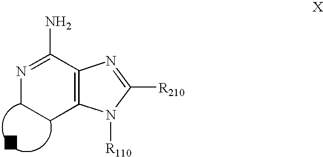

wherein

A is ═N—CR═CR—CR═; ═CR—N═CR—CR═; ═CR—CR═N—CR═; or ═CR—CR═CR—N═;

R₁₁₀ is selected from:

-hydrogen;

—C₁₋₂₀ alkyl or C₂₋₂₀ alkenyl that is unsubstituted or substituted by one or more substituents selected from:
-aryl;
-heteroaryl;
-heterocyclyl;
—O—C₁₋₂₀alkyl;
—O—(C₁₋₂₀ alkyl)₀₋₁-aryl;
—O—(C₁₋₂₀ alkyl)₀₋₁-heteroaryl;
—O—(C₁₋₂₀ alkyl)₀₋₁-heterocyclyl;
—CO—O—C₁₋₂₀ alkyl;
—S(O)₀₋₂—C₁₋₂₀ alkyl;
—S(O)₀₋₂—(C₁₋₂₀ alkyl)₀₋₁-aryl;
—S(O)₀₋₂—(C₁₋₂₀ alkyl)₀₋₁heteroaryl;
—S(O)₀₋₂—(C₁₋₂₀ alkyl)₀₋₁heterocyclyl;
—N(R₃₁₀)₂;
—N₃;
oxo;
-halogen;
—NO₂;
—OH; and
—SH; and

—C₁₋₂₀ alkyl-NR₃₁₀-Q-X—R₄₁₀ or —C₂₋₂₀ alkenyl-NR₃₁₀-Q-X—R₄₁₀ wherein Q is —CO— or —SO₂—; X is a bond, —O— or —NR₃₁₀- and R₄₁₀ is aryl; heteroaryl; heterocyclyl; or —C₁₋₂₀ alkyl or C₂₋₂₀ alkenyl that is unsubstituted or substituted by one or more substituents selected from:
-aryl;
-heteroaryl;
-heterocyclyl;
—O—C₁₋₂₀ alkyl;
—O—(C₁₋₂₀ alkyl)₀₋₁-aryl;
—O—(C₁₋₂₀ alkyl)₀₋₁-heteroaryl;
—O—(C₁₋₂₀ alkyl)₀₋₁-heterocyclyl;
—CO—O—C₁₋₂₀ alkyl;
—S(O)₀₋₂—C₁₋₂₀ alkyl;
—S(O)₀₋₂—(C₁₋₂₀ alkyl)₀₋₁aryl;
—S(O)₀₋₂—(C₁₋₂₀ alkyl)₀₋₁-heteroaryl;
—S(O)₀₋₂—(C₁₋₂₀ alkyl)₀₋₁-heterocyclyl;
—N(R₃₁₀)₂;

—NR₃₁₀—CO—O—C₁₋₂₀ alkyl;
—N₃;
oxo;
-halogen;
—NO₂;
—OH; and
—SH; or R₄₁₀ is

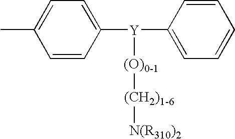

wherein Y is —N— or —CR—;
   R₂₁₀ is selected from:
-hydrogen;
—C₁₋₁₀ alkyl;
—C₂₋₁₀ alkenyl;
-aryl;
—C₁₋₁₀ alkyl-O—C₁₋₁₀ alkyl;
—C₁₋₁₀ alkyl-O—C₂₋₁₀ alkenyl; and
—C₁₋₁₀ alkyl or C₂₋₁₀ alkenyl substituted by one or more substituents selected from:
   —OH;
   -halogen;
   —N(R₃₁₀)₂;
   —CO—N(R₃₁₀)₂;
   —CO—C₁₋₁₀ alkyl;
   —N₃;
   -aryl;
   -heteroaryl;
   -heterocyclyl;
   —CO-aryl; and
   —CO-heteroaryl;

each R₃₁₀ is independently selected from hydrogen and C₁₋₁₀ alkyl; and

each R is independently selected from hydrogen, C₁₋₁₀ alkyl, C₁₋₁₀ alkoxy, halogen and trifluoromethyl;

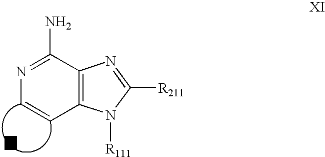

wherein

B is —NR—C(R)₂—C(R)₂—C(R)₂—; —C(R)₂—NR—C(R)₂—C(R)₂—;

### —C(R)₂—C(R)₂—NR—C(R)₂— or —C(R)₂—C(R)₂—C(R)₂—NR—;

R₁₁₁ is selected from:

-hydrogen;

—C₁₋₂₀ alkyl or C₂₋₂₀ alkenyl that is unsubstituted or substituted by one or more substituents selected from:
-aryl;
-heteroaryl;
-heterocyclyl;
—O—C₁₋₂₀ alkyl;
—O—(C₁₋₂₀ alkyl)₀₋₁aryl;
—O—(C₁₋₂₀ alkyl)₀₋₁-heteroaryl;
—O—(C₁₋₂₀ alkyl)₀₋₁-heterocyclyl;
—CO—O—C₁₋₂₀ alkyl;
—S(O)₀₋₂—C₁₋₂₀ alkyl;
—S(O)₀₋₂₀—(C₁₋₂₀ alkyl)₀₋₁-aryl;
—S(O)₀₋₂—(C₁₋₂₀ alkyl)₀₋₁-heteroaryl;
—S(O)₀₋₂₀—(C₁₋₂₀ alkyl)₀₋₁-heterocyclyl;
—N(R₃₁₁)₂;
—N₃;
oxo;
-halogen;
—NO₂;
—OH; and
—SH; and

—C₁₋₂₀ alkyl-NR₃₁₁-Q-X—R₄₁₁ or —C₂₋₂₀ alkenyl-NR₃₁₁-Q-X—R₄₁₁ wherein Q is —CO— or —SO₂—; X is a bond, —O— or —NR₃₁₁- and R₄₁₁ is aryl; heteroaryl; heterocyclyl; or —C₁₋₂₀ alkyl or C₂₋₂₀ alkenyl that is unsubstituted or substituted by one or more substituents selected from:
-aryl;
-heteroaryl;
-heterocyclyl;
—O—C₁₋₂₀alkyl;
—O—(C₁₋₂₀ alkyl)₀₋₁-aryl;
—O—(C₁₋₂₀ alkyl)₀₋₁heteroaryl;
—O—(C₁₋₂₀ alkyl)₀₋₁-heterocyclyl;
—CO—O—C₁₋₂₀ alkyl;
—S(O)₀₋₂₀—C₁₂₀ alkyl;
—S(O)₀₋₂—(C₁₋₂₀ alkyl)₀₋₁aryl;
—S(O)₀₋₂—(C₁₋₂₀ alkyl)₀₋₁heteroaryl;
—S(O)₀₋₂—(C₁₋₂₀ alkyl)₀₋₁-heterocyclyl;
—N(R₃₁₁)₂;
—NR₃₁₁—CO—O—C₁₋₂₀alkyl;
—N₃;
oxo;
-halogen;
—NO₂;
—OH; and
—SH; or R₄₁₁ is

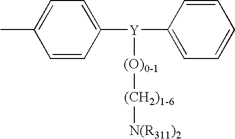

wherein Y is —N— or —CR—;
   R₂₁₁ is selected from:
-hydrogen;
—C₁₋₁₀ alkyl;
—C₂₋₁₀ alkenyl;
-aryl;
—C₁₋₁₀ alkyl —O—C₁₋₁₀-alkyl;
—C₁₋₁₀ alkyl-O—C₂₋₁₀ alkenyl; and
—C₁₋₁₀ alkyl or C₂₋₁₀ alkenyl substituted by one or more substituents selected from:
   —OH;
   -halogen;
   —N(R₃₁₁)₂;
   —CO—N(R₃₁₁)₂;
   —CO—C₁₋₁₀ alkyl;
   —N₃;
   -aryl;
   -heteroaryl;
   -heterocyclyl;
   —CO-aryl; and
   —CO-heteroaryl;

each R₃₁₁ is independently selected from hydrogen and C₁₋₁₀ alkyl; and

each R is independently selected from hydrogen, C₁₋₁₀ alkyl, C₁₋₁₀ alkoxy, halogen, and trifluoromethyl;

and pharmaceutically acceptable salts thereof.

In another embodiment, the IRM compound can be chosen from 1H-imidazo[4,5-c]quinolin-4-amines and tetrahydro-1H-imidazo[4,5-c]quinolin-4-amines defined by Formulas XII, XIII and XIV below:

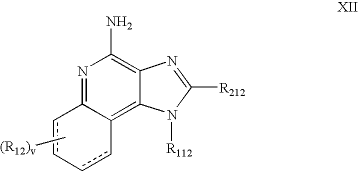

wherein

R₁₁₂ is -alkyl-NR₃₁₂—CO—R₄₁₂ or -alkenyl-NR₃₁₂—CO—R₄₁₂ wherein R₄₁₂ is aryl, heteroaryl, alkyl or alkenyl, each of which may be unsubstituted or substituted by one or more substituents selected from:
-alkyl;
-alkenyl;
-alkynyl;
-(alkyl)₀₋₁-aryl;
-(alkyl)₀₋₁-(substituted aryl);
-(alkyl)₀₋₁-heteroaryl;
-(alkyl)₀₋₁-(substituted heteroaryl);
—O-alkyl;
—O-(alkyl)₀₋₁-aryl;
—O-(alkyl)₀₋₁-(substituted aryl);
—O-(alkyl)₀₋₁-heteroaryl;
—O-(alkyl)₀₋₁-(substituted heteroaryl);
—CO-aryl;
—CO-(substituted aryl);
—CO-heteroaryl;
—CO-(substituted heteroaryl);
—COOH;
—CO—O-alkyl;
—CO-alkyl;
—S(O)₀₋₂-alkyl;
—S(O)₀₋₂-(alkyl)₀₋₁aryl;
—S(O)₀₋₂-(alkyl)₀₋₁-(substituted aryl);
—S(O)₀₋₂-(alkyl)₀₋₁-heteroaryl;
—S(O)₀₋₁-(alkyl)₀₋₁-(substituted heteroaryl);
—P(O)(OR₃₁₂)₂;
—NR₃₁₂—CO—O-alkyl;
—N₃;
-halogen;
—NO₂;
—CN;
-haloalkyl;
—O-haloalkyl;
—CO-haloalkyl;
—OH;
—SH; and in the case that R₄₁₂ is alkyl, alkenyl, or heterocyclyl, oxo;
or R₄₁₂ is

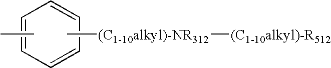

wherein R₅₁₂ is an aryl, (substituted aryl), heteroaryl, (substituted heteroaryl), heterocyclyl or (substituted heterocyclyl) group;

R₂₁₂ is selected from:
-hydrogen;
-alkyl;
-alkenyl;
-aryl;
-(substituted aryl);
-heteroaryl;
-(substituted heteroaryl);
-heterocyclyl;
-(substituted heterocyclyl);
-alkyl-O-alkyl;
-alkyl-O-alkenyl; and
-alkyl or alkenyl substituted by one or more substituents selected from:
   —OH;
   -halogen;
   —N(R₃₁₂)₂;
   —CO—N(R₃₁₂)₂;
   —CO—C₁₋₁₀ alkyl;
   —CO—O—C₁₋₁₀ alkyl;
   —N₃;
   -aryl;
   -(substituted aryl);
   -heteroaryl;
   -(substituted heteroaryl);
   -heterocyclyl;
   -(substituted heterocyclyl);
   —CO-aryl; and
   —CO-heteroaryl;

each R₃₁₂ is independently selected from hydrogen; C₁₋₁₀ alkyl-heteroaryl; C₁₋₁₀ alkyl-(substituted heteroaryl); C₁₋₁₀ alkyl-aryl; C₁₋₁₀ alkyl-(substituted aryl) and C₁₋₁₀ alkyl;

v is 0 to 4;

and each R₁₂ present is independently selected from C₁₋₁₀ alkyl, C₁₋₁₀ alkoxy, halogen, and trifluoromethyl;

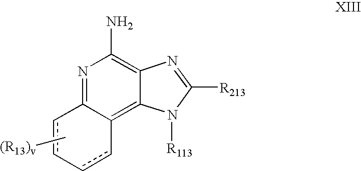

wherein

R₁₁₃ is -alkyl-NR₃₁₃—SO₂—X—R₄₁₃ or -alkenyl-NR₃₁₃—SO₂—X—R₄₁₃;

X is a bond or —NR₅₁₃—;

R₄₁₃ is aryl, heteroaryl, heterocyclyl, alkyl or alkenyl, each of which may be unsubstituted or substituted by one or more substituents selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkyl)₀₋₁aryl;
—O-(alkyl)₀₋₁-substituted aryl;
—O-(alkyl)₀₋₁-heteroaryl;
—O-(alkyl)₀₋₁-substituted heteroaryl;
—O-(alkyl)₀₋₁-heterocyclyl;
—O-(alkyl)₀₋₁-substituted heterocyclyl;
—COOH;
—CO—O-alkyl;
—CO-alkyl;
—S(O)₀₋₂-alkyl;
—S(O)₀₋₂-(alkyl)₀₋₁-aryl;
—S(O)₀₋₂-(alkyl)₀₋₁-substituted aryl;
—S(O)₀₋₂-(alkyl)₀₋₁-heteroaryl;
—S(O)₀₋₂-(alkyl)₀₋₁-substituted heteroaryl;
—S(O)₀₋₂-(alkyl)₀₋₁-heterocyclyl;
—S(O)₀₋₂-(alkyl)₀₋₁-substituted heterocyclyl;
-(alkyl)₀₋₁-NR₃₁₃R₃₁₃;
-(alkyl)₀₋₁-NR₃₁₃—CO—O-alkyl;
-(alkyl)₀₋₁-NR₃₁₃—CO-alkyl;
-(alkyl)₀₋₁-NR₃₁₃—CO-aryl;
-(alkyl)₀₋₁-NR₃₁₃—CO-substituted aryl;
-(alkyl)₀₋₁-NR₃₁₃—CO-heteroaryl;
-(alkyl)₀₋₁-NR₃₁₃—CO-substituted heteroaryl;
—N₃;
-halogen;
-haloalkyl;
-haloalkoxy;
—CO-haloalkyl;
—CO-haloalkoxy;
—NO₂;
—CN;
—OH;
—SH; and in the case that R₄₁₃ is alkyl, alkenyl, or heterocyclyl, oxo;

R₂₁₃ is selected from:
-hydrogen;
-alkyl;
-alkenyl;
-aryl;
-substituted aryl;
-heteroaryl;
-substituted heteroaryl;
-alkyl-O-alkyl;
-alkyl-O-alkenyl; and
alkyl or alkenyl substituted by one or more substituents selected from:
   —OH;
   -halogen;
   —N(R₃₁₃)₂;
   —CO—N(R₃₁₃)₂;
   —CO—C₁₋₁₀ alkyl;
   —CO—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —CO-aryl;
   —CO-(substituted aryl);
   —CO-heteroaryl; and
   —CO-(substituted heteroaryl);

each R₃₁₃ is independently selected from hydrogen and C₁₋₁₀ alkyl; or when X is a bond R₃₁₃ and R₄₁₃ can join to form a 3 to 7 membered heterocyclic or substituted heterocyclic ring;

R₅₁₃ is selected from hydrogen and C₁₋₁₀ alkyl, or R₄₁₃ and R₅₁₃ can combine to form a 3 to 7 membered heterocyclic or substituted heterocyclic ring;

v is 0 to 4;

and each R₁₃ present is independently selected from C₁₋₁₀ alkyl, C₁₋₁₀ alkoxy, halogen, and trifluoromethyl;

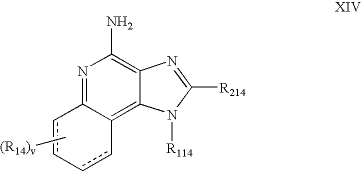

wherein
R₁₁₄ is -alkyl-NR₃₁₄—CY—NR₅₁₄—X—R₄₁₄ or
-alkenyl-NR₃₁₄—CY—NR₅₁₄—X—R₄₁₄
   wherein

Y is ═O or ═S;

X is a bond, —CO— or —SO₂—;

R₄₁₄ is aryl, heteroaryl, heterocyclyl, alkyl or alkenyl, each of which may be unsubstituted or substituted by one or more substituents selected from:
-alkyl;
-alkenyl;
-aryl;
-heteroaryl;
-heterocyclyl;
-substituted aryl;
-substituted heteroaryl;
-substituted heterocyclyl;
—O-alkyl;
—O-(alkyl)₀₋₁aryl;
—O-(alkyl)₀₋₁-substituted aryl;
—O-(alkyl)₀₋₁-heteroaryl;
—O-(alkyl)₀₋₁-substituted heteroaryl;
—O-(alkyl)₀₋₁-heterocyclyl;
—O-(alkyl)₀₋₁-substituted heterocyclyl;
—COOH;
—CO—O-alkyl;
—CO-alkyl;
—S(O)₀₋₂-alkyl;
—S(O)₀₋₂-(alkyl)₀₋₁aryl;
—S(O)₀₋₂-(alkyl)₀₋₁-substituted aryl;
—S(O)₀₋₂-(alkyl)₀₋₁heteroaryl;
—S(O)O-2-(alkyl)₀₋₁-substituted heteroaryl;
—S(O)₀₋₂-(alkyl)₀₋₁heterocyclyl;
—S(O)₀₋₂-(alkyl)₀₋₁-substituted heterocyclyl;
-(alkyl)₀₋₁-NR₃₁₄R₃₁₄;
-(alkyl)₀₋₁-NR₃₁₄—CO—O-alkyl;
-(alkyl)₀₋₁-NR₃₁₄—CO-alkyl;
-(alkyl)₀₋₁-NR₃₁₄—CO-aryl;
-(alkyl)₀₋₁-NR₃₁₄—CO-substituted aryl;
-(alkyl)₀₋₁-NR₃₁₄—CO-heteroaryl;
-(alkyl)₀₋₁-NR₃₁₄—CO-substituted heteroaryl;
—N₃;
-halogen;
-haloalkyl;
-haloalkoxy;
—CO-halo alkoxy;
—NO₂;
—CN;
—OH;
—SH; and, in the case that R₄₁₄ is alkyl, alkenyl or heterocyclyl, oxo;

with the proviso that when X is a bond R₄₁₄ can additionally be hydrogen;

R₂₁₄ is selected from:
-hydrogen;
-alkyl;
-alkenyl;
-aryl;
-substituted aryl;
-heteroaryl;
-substituted heteroaryl;
-alkyl-O-alkyl;
-alkyl-O-alkenyl; and
alkyl or alkenyl substituted by one or more substituents selected from:
   —OH;
   -halogen;
   —N(R₃₁₄)₂;
   —CO—N(R₃₁₄)₂;
   —CO—C₁₋₁₀ alkyl;
   —CO—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —CO-aryl;
   —CO-(substituted aryl);
   —CO-heteroaryl; and
   —CO-(substituted heteroaryl);

each R₃₁₄ is independently selected from hydrogen and C₁₋₁₀ alkyl;

R₅₁₄ is selected from hydrogen and C₁₋₁₀ alkyl, or R₄₁₄ and R₅₁₄ can combine to form a 3 to 7 membered heterocyclic or substituted heterocyclic ring;

v is 0 to 4;

and each R₁₄ present is independently selected from C₁₋₁₀ alkyl, C₁₋₁₀ alkoxy, halogen, and trifluoromethyl;

and pharmaceutically acceptable salts thereof.

In another embodiment, the IRM compound can be chosen from 1H-imidazo[4,5-c]quinolin-4-amines and tetrahydro-1H-imidazo[4,5-c]quinolin-4-amines defined by Formulas XV, XVI, XVII, XVIII, XIX, XX, XXI, XXII, XXIII, XXIV, XXV, and XXVI below:

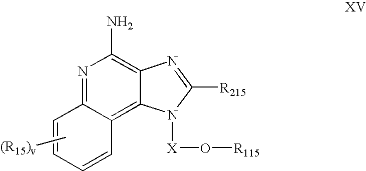

wherein:

X is —CHR₅₁₅—, —CHR₅₁₅-alkyl-, or —CHR₅₁₅-alkenyl-;

R₁₁₅ is selected from:
—R₄₁₅—CR₃₁₅-Z-R₆₁₅-alkyl;
—R₄₅—CR₃₁₅-Z-R₆₁₅-alkenyl;
—R₄₁₅—CR₃₁₅-Z-R₆₁₅-aryl;
—R₄₁₅—CR₃₁₅-Z-R₆₁₅-heteroaryl;
—R₄₁₅—CR₃₁₅-Z-R₆₁₅-heterocyclyl;
—R₄₁₅—CR₃₁₅-Z-H;
—R₄₁₅—NR₇₁₅—CR₃₁₅—R₆₁₅-alkyl;
—R₄₁₅—NR₇₁₅—CR₃₁₅—R₆₁-alkenyl;
—R₄₁₅—NR₇₁₅—CR₃₁₅—R₆₁₅-aryl;
—R₄₁₅—NR₇₁₅—CR₃₁₅—R₆₁₅-heteroaryl;
—R₄₁₅—NR₇₁₅—CR₃₁₅—R₆₁₅-heterocyclyl; and
—R₄₁₅—NR₇₁₅—CR₃₁₅—R₈₁₅;

Z is —NR₅₁₅—, —O—, or —S—;

R₂₁₅ is selected from:
-hydrogen;
-alkyl;
-alkenyl;
-aryl;
-heteroaryl;
-heterocyclyl;
-alkyl-Y-alkyl;
-alkyl-Y-alkenyl;
-alkyl-Y-aryl; and
-alkyl or alkenyl substituted by one or more substituents selected from:
   —OH;
   -halogen;
   —N(R₅₁₅)₂;
   —CO—N(R₅₁₅)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

R₃₁₅ is ═O or ═S;

R₄₁₅ is alkyl or alkenyl, which may be interrupted by one or more —O— groups;

each R₅₁₅ is independently H or C₁₋₁₀ alkyl;

R₆₁₅ is a bond, alkyl, or alkenyl, which may be interrupted by one or more —O— groups;

R₇₁₅ is H, C₁₀ alkyl, or arylalkyl; or R₄₁₅ and R₇₁₅ can join together to form a ring;

R₈₁₅ is H or C₁₋₁₀ alkyl; or R₇₁₅ and R₈₁₅ can join together to form a ring;

Y is —O— or —S(O)₀₋₂—;

v is 0 to 4; and

each R₁₅ present is independently selected from C₁₋₁₀ alkyl, C₁₋₁₀ alkoxy, hydroxy, halogen, and trifluoromethyl;

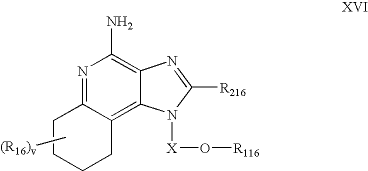

wherein:

X is —CHR₅₁₆—, —CHR₅₁₆-alkyl-, or —CHR₅₁₆-alkenyl-;

R₁₁₆ is selected from:
—R₄₁₆—CR₃₁₆-Z-R₆₁₆-alkyl;
—R₄₁₆—CR₃₁₆-Z-R₆₁₆-alkenyl;
—R₄₁₆—CR₃₁₆-Z-R₆₁₆-aryl;
—R₄₁₆—CR₃₁₆-Z-R₆₁₆-heteroaryl;
—R₄₁₆—CR₃₁₆-Z-R₆₁₆-heterocyclyl;
—R₄₁₆—CR₃₁₆-Z-H;
—R₄₁₆—NR₇₁₆—CR₃₁₆—R₆₁₆-alkyl;
—R₄₁₆—NR₇₁₆—CR₃₁₆—R₆₁₆-alkenyl;
—R₄₁₆—NR₇₁₆—CR₃₁₆—R₆₁₆-aryl;
—R₄₁₆—NR₇₁₆—CR₃₁₆—R₈₁₆-heteroaryl;
—R₄₁₆—NR₇₁₆—CR₃₁₆—R₈₁₆-heterocyclyl; and
—R₄₁₆—NR₇₁₆—CR₃₁₆—R₈₁₆;

Z is —NR₅₁₆—, —O—, or —S—;

R₂₁₆ is selected from:
-hydrogen;
-alkyl;
-alkenyl;
-aryl;
-heteroaryl;
-heterocyclyl;
-alkyl-Y-alkyl;
-alkyl-Y-alkenyl;
-alkyl-Y-aryl; and
-alkyl or alkenyl substituted by one or more substituents selected from:
   —OH;
   -halogen;
   —N(R₅₁₆)₂;
   —CO—N(R₅₁₆)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

R₃₁₆ is ═O or ═S;

R₄₁₆ is alkyl or alkenyl, which may be interrupted by one or more —O— groups;

each R₅₁₆ is independently H or C₁₋₁₀ alkyl;

R₆₁₆ is a bond, alkyl, or alkenyl, which may be interrupted by one or more —O— groups;

R₇₁₆ is H, C₁₋₁₀ alkyl, arylalkyl; or R₄₁₆ and R₇₁₆ can join together to form a ring;

R₈₁₆ is H or C₁₋₁₀ alkyl; or R₇₁₆ and R₈₁₆ can join together to form a ring;

Y is —O— or —S(O)₀₋₂—;

v is 0 to 4; and

each R₁₆ present is independently selected from C₁₋₁₀ alkyl, C₁₋₁₀ alkoxy, hydroxy, halogen, and trifluoromethyl;

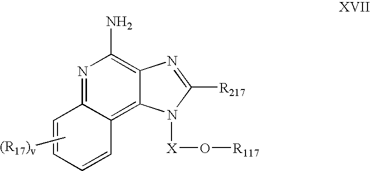

wherein:

X is —CHR₃₁₇—, —CHR₃₁₇-alkyl-, or —CHR₃₁₇-alkenyl-;

R₁₁₇ is selected from:
-alkenyl;
-aryl; and
—R₄₁₇-aryl;

R₂₁₇ is selected from:
-hydrogen;
-alkyl;
-alkenyl;
-aryl;
-heteroaryl;
-heterocyclyl;
-alkyl-Y-alkyl;
-alkyl-Y-alkenyl;
-alkyl-Y-aryl; and
-alkyl or alkenyl substituted by one or more substituents selected from:
   —OH;
   -halogen;
   —N(R₃₁₇)₂;
   —CO—N(R₃₁₇)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

R₄₁₇ is alkyl or alkenyl, which may be interrupted by one or more —O— groups;

each R₃₁₇ is independently H or C₁₋₁₀ alkyl;

each Y is independently —O— or —S(O)₀₋₂—;

v is 0 to 4; and

each R₁₇ present is independently selected from C₁₋₁₀ alkyl, C₁₋₁₀ alkoxy, hydroxy, halogen, and trifluoromethyl;

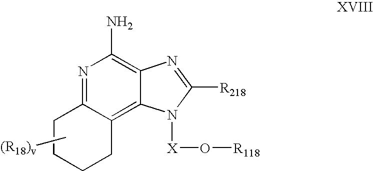

wherein:

X is —CHR₃₁₈—, —CHR₃₁₈-alkyl-, or —CHR₃₁₈-alkenyl-;

R₁₈ is selected from:
-aryl;
-alkenyl; and
—R₄₁₈-aryl;

R₂₁₈ is selected from:
-hydrogen;
-alkyl;
-alkenyl;
-aryl;
-heteroaryl;
-heterocyclyl;
-alkyl-Y-alkyl;
-alkyl-Y-aryl;
alkyl-Y-alkenyl; and
-alkyl or alkenyl substituted by one or more substituents selected from:
   —OH;
   -halogen;
   —N(R₃₁₈)₂;
   —CO—N(R₃₁₈)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

R₄₁₈ is alkyl or alkenyl, which may be interrupted by one or more —O— groups;

each R₃₁₈ is independently H or C₁₋₁₀ alkyl;

each Y is independently —O— or —S(O)₀₋₂—;

v is 0 to 4; and

each R₁₈ present is independently selected C₁₋₁₀ alkyl, C₁₋₁₀ alkoxy, hydroxy, halogen, and trifluoromethyl;

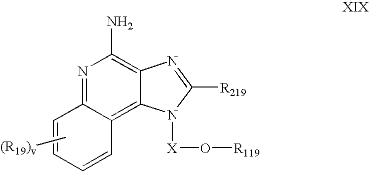

wherein:

X is —CHR₃₁₉—, —CHR₃₁₉-alkyl-, or —CHR₃₁₉-alkenyl-;

R₁₁₉ is selected from:
-heteroaryl;
-heterocyclyl;
—R₄₁₉— heteroaryl; and
—R₄₁₉-heterocyclyl;

R₂₁₉ is selected from:
-hydrogen;
-alkyl;
-alkenyl;
-aryl;
-heteroaryl;
-heterocyclyl;
-alkyl-Y-alkyl;
-alkyl-Y-alkenyl;
-alkyl-Y-aryl; and
-alkyl or alkenyl substituted by one or more substituents selected from:
   —OH;
   -halogen;
   —N(R₃₁₉)₂;
   —CO—N(R₃₁₉)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

R₄₁₉ is alkyl or alkenyl, which may be interrupted by one or more —O— groups;

each R₃₁₉ is independently H or C₁₋₁₀ alkyl;

each Y is independently —O— or —S(O)₀₋₂—;

v is 0 to 4; and

each R₁₉ present is independently selected from C₁₋₁₀ alkyl, C₁₋₁₀ alkoxy, hydroxy, halogen, and trifluoromethyl;

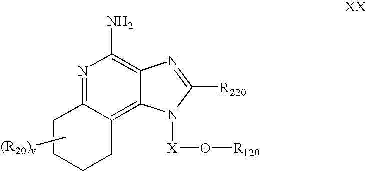

wherein:

X is —CHR₃₂₀—, —CHR₃₂₀-alkyl-, or —CHR₃₂₀-alkenyl-;

R₁₂₀ is selected from:
-heteroaryl;
-heterocyclyl;
—R₄₂₀— heteroaryl; and
—R₄₂₀-heterocyclyl;

R₂₂₀ is selected from:
-hydrogen;
-alkyl;
-alkenyl;
-aryl;
-heteroaryl;
-heterocyclyl;
-alkyl-Y-alkyl;
-alkyl-Y-alkenyl;
-alkyl-Y-aryl; and
-alkyl or alkenyl substituted by one or more substituents selected from:
   —OH;
   -halogen;
   —N(R₃₂₀)₂;
   —CO—N(R₃₂₀)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

R₄₂₀ is alkyl or alkenyl, which may be interrupted by one or more —O— groups;

each R₃₂₀ is independently H or C₁₋₁₀ alkyl;

each Y is independently —O— or —S(O)₀₋₂—;

v is 0 to 4; and

each R₂₀ present is independently selected from C₁₋₁₀ alkyl, C₁₋₁₀ alkoxy, hydroxy, halogen, and trifluoromethyl;

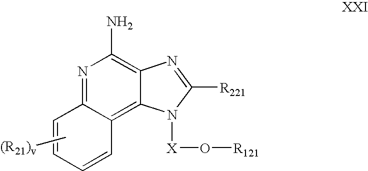

wherein:

X is —CHR₅₂₁—, —CHR₅₂₁-alkyl-, or —CHR₅₂₁-alkenyl-;

R₁₂₁ is selected from:
—R₄₂₁—NR₃₂₁—SO₂—R₆₂₁-alkyl;
—R₄₂₁—NR₃₂—SO₂—R₆₂₁-alkenyl;
—R₄₂₁—NR₃₂₁—SO₂—R₆₂₁-aryl;
—R₄₂₁—NR₃₂₁—SO₂—R₆₂₁-heteroaryl;
—R₄₂₁—NR₃₂₁—SO₂—R₇₂₁-heterocyclyl;
—R₄₂₁—NR₃₂₁—SO₂—R₇₂₁;
—R₄₂₁—NR₃₂₁—SO₂—NR₅₂₁—R₆₂₁-alkyl;
—R₄₂₁—NR₃₂₁—SO₂—NR₅₂₁—R₆₂₁-alkenyl;
—R₄₂₁—NR₃₂₁—SO₂—NR₅₂₁—R₆₂₁-aryl;
—R₄₂₁—NR₃₂₁—SO₂—NR₅₂₁—R₆₂₁-heteroaryl;
—R₄₂₁—NR₃₂₁—SO₂—NR₅₂₁—R₆₂₁-heterocyclyl; and
—R₄₂₁—NR₃₂₁—SO₂—NH₂;

R₂₂₁ is selected from:
-hydrogen;
-alkyl;
-alkenyl;
-aryl;
-heteroaryl;
-heterocyclyl;
-alkyl-Y-alkyl;
-alkyl-Y-alkenyl;
-alkyl-Y-aryl; and
-alkyl or alkenyl substituted by one or more substituents selected from:
   —OH;
   -halogen;
   —N(R₅₂₁)₂;
   —CO—N(R₅₂₁)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

Y is —O— or —S(O)₀₋₂—;

R₃₂₁ is H, C₁₋₁₀ alkyl, or arylalkyl;

each R₄₂₁ is independently alkyl or alkenyl, which may be interrupted by one or more —O— groups; or R₃₂₁ and R₄₂₁ can join together to form a ring;

each R₅₂₁ is independently H, C₁₋₁₀ alkyl, or C₂₋₁₀ alkenyl;

R₆₂₁ is a bond, alkyl, or alkenyl, which may be interrupted by one or more —O— groups;

R₇₂₁ is C₁₋₁₀ alkyl; or R₃₂₁ and R₇₂₁ can join together to form a ring;

v is 0 to 4; and

each R₂₁ present is independently selected from C₁₋₁₀ alkyl, C₁₋₁₀ alkoxy, hydroxy, halogen, and trifluoromethyl;

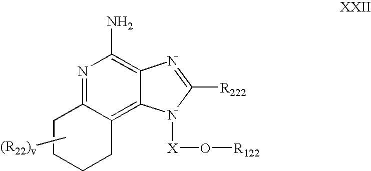

wherein:

X is —CHR₅₂₂—, —CHR₅₂₂-alkyl-, or —CHR₅₂₂-alkenyl-;

R₁₂₂ is selected from:
R₄₂₂—NR₃₂₂—SO₂—R₆₂₂-alkyl;
—R₄₂₂—NR₃₂₂—SO₂—R₆₂₂-alkenyl;
—R₄₂₂—NR₃₂₂—SO₂—R₆₂₂-aryl;
—R₄₂₂—NR₃₂₂—SO₂—R₆₂₂-heteroaryl;
—R₄₂₂—NR₃₂₂—SO₂—R₂₂-heterocyclyl;
—R₄₂₂—NR₃₂₂—SO₂—R₇₂₂;
—R₄₂₂—NR₃₂₂—SO₂—NR₅₂₂—R₆₂₂-alkyl;
—R₄₂₂—NR₃₂₂—SO₂—NR₅₂₂—R₆₂₂-alkenyl;
—R₄₂₂—NR₃₂₂—SO₂—NR₅₂₂—R₆₂₂-aryl;
—R₄₂₂—NR₃₂₂—SO₂—NR₅₂₂—R₆₂₂-heteroaryl;
—R₄₂₂—NR₃₂₂—SO₂—NR₅₂₂—R₆₂₂-heterocyclyl; and
—R₄₂₂—NR₃₂₂—SO₂—NH₂;

—R₂₂₂ is selected from:
-hydrogen;
-alkyl;
-alkenyl;
-aryl;
-heteroaryl;
-heterocyclyl;
-alkyl-Y-alkyl;
-alkyl-Y-alkenyl;
-alkyl-Y-aryl; and
-alkyl or alkenyl substituted by one or more substituents selected from:
   —OH;
   -halogen;
   —N(R₅₂₂)₂;
   —CO—N(R₅₂₂)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

Y is —O— or —S(O)₀₋₂—;

R₃₂₂ is H, C₁₋₁₀ alkyl, or arylalkyl;

each R₄₂₂ is independently alkyl or alkenyl, which may be interrupted by one or more —O— groups; or R₃₂₂ and R₄₂₂ can join together to form a ring;

each R₅₂₂ is independently H, C₁₋₁₀ alkyl, or C₂₋₁₀ alkenyl;

R₆₂₂ is a bond, alkyl, or alkenyl, which may be interrupted by one or more —O— groups;

R₇₂₂ is C₁₋₁₀ alkyl; or R₃₂₂ and R₇₂₂ can join together to form a ring;

v is 0 to 4; and

each R₂₂ present is independently selected from C₁₋₁₀ alkyl, C₁₋₁₀ alkoxy, hydroxy, halogen, and trifluoromethyl;

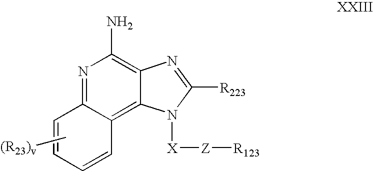

wherein:

X is —CHR₃₂₃—, —CHR₃₂₃-alkyl-, or —CHR₃₂₃-alkenyl-;

Z is —S—, —SO—, or —SO₂—;

R₁₂₃ is selected from:
-alkyl;
-aryl;
-heteroaryl;
-heterocyclyl;
-alkenyl;
—R₄₂₃-aryl;
—R₄₂₃— heteroaryl; and
—R₄₂₃-heterocyclyl;

R₂₂₃ is selected from:
-hydrogen;
-alkyl;
-alkenyl;
-aryl;
-heteroaryl;
-heterocyclyl;
-alkyl-Y-alkyl;
alkyl-Y-alkenyl;
-alkyl-Y-aryl; and
alkyl or alkenyl substituted by one or more substituents selected from:
   —OH;
   -halogen;
   —N(R₃₂₃)₂;
   —CO—N(R₃₂₃)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

each R₃₂₃ is independently H or C₁₋₁₀ alkyl;

each R₄₂₃ is independently alkyl or alkenyl;

each Y is independently —O— or —S(O)₀₋₂—;

v is 0 to 4; and

each R₂₃ present is independently selected from C₁₋₁₀ alkyl, C₁₋₁₀ alkoxy, hydroxy, halogen, and trifluoromethyl;

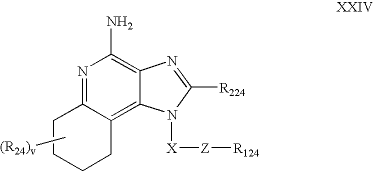

wherein:

X is —CHR₃₂₄—, —CHR₃₂₄-alkyl-, or —CHR₃₂₄-alkenyl-;

Z is —S—, —SO—, or —SO₂—;

R₁₂₄ is selected from:
-alkyl;
-aryl;
-heteroaryl;
-heterocyclyl;
-alkenyl;
—R₂₄-aryl;
—R₂₄— heteroaryl; and
—R₂₄-heterocyclyl;

R₂₂₄ is selected from:
-hydrogen;
-alkyl;
-alkenyl;
-aryl;
-heteroaryl;
-heterocyclyl;
-alkyl-Y-alkyl;
alkyl-Y-alkenyl;
-alkyl-Y-aryl; and
-alkyl or alkenyl substituted by one or more substituents selected from:
   —OH;
   -halogen;
   —N(R₃₂₄)₂;
   —CO—N(R₃₂₄)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

each R₃₂₄ is independently H or C₁₋₁₀ alkyl;

each R₄₂₄ is independently alkyl or alkenyl;

each Y is independently —O— or —S(O)₀₋₂—;

v is 0 to 4; and

each R₂₄ present is independently selected from C₁₋₁₀ alkyl, C₁₋₁₀ alkoxy, hydroxy, halogen, and trifluoromethyl;

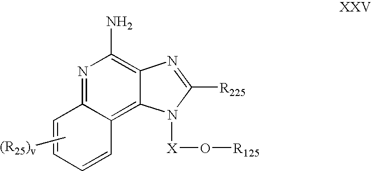

wherein:

X is —CHR₅₂₅—, —CHR₅₂₅-alkyl-, or —CHR₅₂₅-alkenyl-;

R₁₂₅ is selected from:
—R₄₂₅—NR₈₂₅—CR₃₂₅—NR₅₂₅-Z-R₆₂₅-alkyl;
—R₄₂₅—NR₉₂₅—CR₃₂₅—NR₅₂₅-Z-R₆₂₅-alkenyl;
—R₄₂₅—NR₅₂₅—CR₃₂₅—NR₅₂₅-Z-R₆₂₅-aryl;
—R₄₂₅—NR₈₂₅—CR₃₂₅—NR₅₂₅-Z-R₆₂₅-heteroaryl;
—R₄₂₅—NR₈₂₅—CR₃₂₅—NR₅₂₅-Z-R₆₂₅-heterocyclyl;
—R₄₂₅—NR₅₂₅—CR₃₂₅—NR₅₂₅R₇₂₅;
—R₄₂₅—NR₈₂₅—CR₃₂₅—NR₉₂₅-Z-R₆₂₅-alkyl;
—R₄₂₅—NR₈₂₅—CR₃₂₅—NR₉₂₅-Z-R₆₂₅-alkenyl;
—R₄₂₅—NR₈₂₅—CR₃₂₅—NR₉₂₅-Z-R₆₂₅-aryl;
—R₄₂₅—NR₈₂₅—CR₃₂₅—NR₉₂₅-Z-R₆₂₅-heteroaryl; and
—R₄₂₅—NR₉₂₅—CR₃₂₅—NR₉₂₅-Z-R₆₂₅-heterocyclyl;

R₂₂₅ is selected from:
-hydrogen;
-alkyl;
-alkenyl;
-aryl;
-heteroaryl;
-heterocyclyl;
-alkyl-Y-alkyl;
-alkyl-Y-alkenyl;
-alkyl-Y-aryl; and
-alkyl or alkenyl substituted by one or more substituents selected from:
   —OH;
   -halogen;
   —N(R₅₂₅)₂;
   —CO—N(R₅₂₅)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

each R₃₂₅ is ═O or ═S;

each R₄₂₅ is independently alkyl or alkenyl, which may be interrupted by one or more —O— groups;

each R₅₂₅ is independently H or C₁₋₁₀ alkyl;

R₆₂₅ is a bond, alkyl, or alkenyl, which may be interrupted by one or more —O— groups;

R₇₂₅ is H or C₁₋₁₀ alkyl which may be interrupted by a hetero atom, or R₇₂₅ can join with R₅₂₅ to form a ring;

R₈₂₅ is H, C₁₋₁₀ alkyl, or arylalkyl; or R₄₂₅ and R₈₂₅ can join together to form a ring;

R₉₂₅ is C₁₋₁₀ alkyl which can join together with R₈₂₅ to form a ring;

each Y is independently —O— or —S(O)₀₋₂—;

Z is a bond, —CO—, or —SO₂—;

v is 0 to 4; and

each R₂₅ present is independently selected C₁₋₁₀ alkyl, C₁₋₁₀ alkoxy, hydroxy, halogen, and trifluoromethyl;

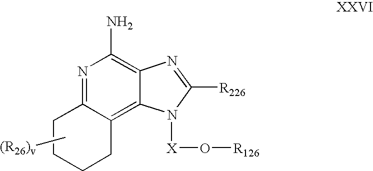

wherein:

X is —CHR₅₂₆—, —CHR₅₂₆-alkyl-, or —CHR₅₂₆-alkenyl-;

R₁₂₆ is selected from:
—R₄₂₆—NR₈₂₆—CR₃₂₆—NR₅₂₆-Z-R₆₂₆-alkyl;
—R₄₂₆—NR₈₂₆—CR₃₂₆—NR₅₂₆-Z-R₆₂₆-alkenyl;
—R₄₂₆—NR₈₂₆—CR₃₂₆—NR₅₂₆-Z-R₆₂₆-aryl;
—R₄₂₆—NR₈₂₆—CR₃₂₆—NR₅₂₆-Z-R₆₂₆-heteroaryl;
—R₄₂₆—NR₈₂₆—CR₃₂₆—NR₅₂₆-Z-R₆₂₆-heterocyclyl;
—R₄₂₆—NR₈₂₆—CR₃₂₆—NR₅₂₆R₇₂₆;
—R₄₂₆—NR₉₂₆—CR₃₂₆—NR₉₂₆-Z-R₆₂₆-alkyl;
—R₄₂₆—NR₉₂₆—CR₃₂₆—NR₉₂₆-Z-R₆₂₆-alkenyl;
—R₄₂₆—NR₈₂₆—CR₃₂₆—NR₉₂₆-Z-R₆₂₆-aryl;
—R₄₂₆—NR₈₂6-CR₃₂₆—NR₉₂₆-Z-R₆₂₆-heteroaryl; and
—R₄₂6-NR₈₂₆—CR₃₂₆—NR₉₂₆-Z-R₆₂₆-heterocyclyl;

R₂₂₆ is selected from:
-hydrogen;
-alkyl;
-alkenyl;
-aryl;
-heteroaryl;
-heterocyclyl;
-alkyl-Y-alkyl;
-alkyl-Y-alkenyl;
-alkyl-Y-aryl; and
-alkyl or alkenyl substituted by one or more substituents selected from:
   —OH;
   -halogen;
   —N(R₅₂₆)₂;
   —CO—N(R₅₂₆)₂;
   —CO—C₁₋₁₀ alkyl;
   —CO—O—C₁₋₁₀ alkyl;
   —N₃;
   -aryl;
   -heteroaryl;
   -heterocyclyl;
   —CO-aryl; and
   —CO-heteroaryl;

each R₃₂₆ is ═O or ═S;

each R₄₂₆ is independently alkyl or alkenyl, which may be interrupted by one or more —O— groups;

each R₅₂₆ is independently H or C₁₋₁₀ alkyl;

R₆₂₆ is a bond, alkyl, or alkenyl, which may be interrupted by one or more —O— groups;

R₇₂₆ is H or C₁₋₁₀ alkyl which may be interrupted by a hetero atom, or R₇₂₆ can join with R₅₂₆ to form a ring;

R₈₂₆ is H, C₁₋₁₀ alkyl, or arylalkyl; or R₄₂₆ and R₈₂₆ can join together to form a ring;

R₉₂₆ is C₁₋₁₀ alkyl which can join together with R₈₂₆ to form a ring;

each Y is independently —O— or —S(O)₀₋₂—;

Z is a bond, —CO—, or —SO₂—;

v is 0 to 4; and

each R₂₆ present is independently selected from C₁₋₁₀ alkyl, C₁₋₁₀ alkoxy, hydroxy, halogen, and trifluoromethyl;

and pharmaceutically acceptable salts of any of the foregoing.

In another embodiment, the IRM compound can be chosen from 1H-imidazo[4,5-c]pyridin-4-amines defined by Formula XXVII below:

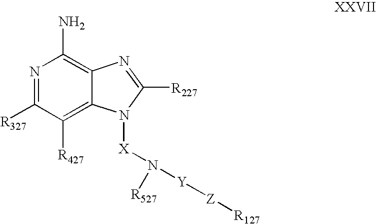

wherein

X is alkylene or alkenylene;

Y is —CO— or —CS;

Z is a bond, —O—, or —S—;

R₁₂₇ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkyl)₀₋₁-aryl;
—O-(alkyl)₀₋₁-(substituted aryl);
—O-(alkyl)₀₋₁-heteroaryl;
—O-(alkyl)₀₋₁-(substituted heteroaryl);
—O-(alkyl)₀₋₁-heterocyclyl;
—O-(alkyl)₀₋₁-(substituted heterocyclyl);
—COOH;
—CO—O-alkyl;
—CO-alkyl;
—S(O)₀₋₂-alkyl;
—S(O)₀₋₂-(alkyl)₀₋₁-aryl;
—S(O)₀₋₂-(alkyl)₀₋₁-(substituted aryl);
—S(O)₀₋₂-(alkyl)₀₋₁heteroaryl;
—S(O)₀₋₂-(alkyl)₀₋₁-(substituted heteroaryl);
—S(O)₀₋₂-(alkyl)₀₋₁-heterocyclyl;
—S(O)₀₋₂-(alkyl)₀₋₁-(substituted heterocyclyl);
-(alkyl)₀₋₁-N(R₆₂₇)₂;
-(alkyl)₀₋₁-NR₆₂₇—CO—O-alkyl;
-(alkyl)₀₋₁-NR₆₂₇—CO-alkyl;
-(alkyl)₀₋₁-NR₆₂₇—CO-aryl;
-(alkyl)₀₋₁-NR₆₂₇—CO-(substituted aryl);
-(alkyl)₀₋₁-NR₆₂₇—CO-heteroaryl;
-(alkyl)₀₋₁-NR₆₂₇—CO-(substituted heteroaryl);
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;

R₂₂₇ is selected from:
-hydrogen;
-alkyl;
-alkenyl;
-aryl;
-substituted aryl;
-heteroaryl;
-substituted heteroaryl;
-alkyl-O-alkyl;
-alkyl-S-alkyl;
-alkyl-O-aryl;
-alkyl-S-aryl:
-alkyl-O-alkenyl;
-alkyl-S-alkenyl; and
-alkyl or alkenyl substituted by one or more substituents selected from:
   —OH;
   -halogen;
   —N(R₆₂₇)₂;
   —CO—N(R₆₂₇)₂;
   —CS—N(R₆₂₇)₂;
   —SO₂—N(R₆₂₇)₂;
   —NR₆₂₇—CO—C₁₋₁₀ alkyl;
   —NR₆₂₇—CS—C₁₋₁₀ alkyl;
   —NR₆₂₇—SO₂—C₁₋₁₀ alkyl;
   —CO—C₁₋₁₀ alkyl;
   —CO—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —CO-aryl;
   —CO-(substituted aryl);
   —CO-heteroaryl; and
   —CO-(substituted heteroaryl);

R₃₂₇ and R₄₂₇ are independently selected from hydrogen, alkyl, alkenyl, halogen, alkoxy, amino, alkylamino, dialkylamino, and alkylthio;

R₅₂₇ is H or C₁₋₁₀ alkyl, or R₅₂₇ can join with X to form a ring that contains one or two heteroatoms; or when R₁₂₇ is alkyl, R₅₂₇ and R₁₂₇ can join to form a ring;

each R₆₂₇ is independently H or C₁₋₁₀alkyl;

and pharmaceutically acceptable salts thereof.

In another embodiment, the IRM compound can be chosen from 1H-imidazo[4,5-c]pyridin-4-amines defined by Formula XXVIII below:

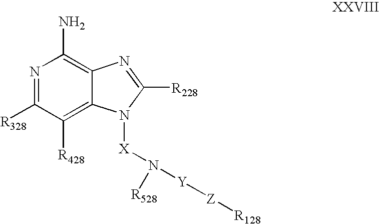

wherein

X is alkylene or alkenylene;

Y is —SO₂—;

Z is a bond or —NR₆₂₈—;

R₁₂₈ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;

O—O-(alkyl)₀₋₁aryl;
—O-(alkyl)₀₋₁-(substituted aryl);
—O-(alkyl)₀₋₁-heteroaryl;
—O-(alkyl)₀₋₁-(substituted heteroaryl);
—O-(alkyl)₀₋₁-heterocyclyl;
—O-(alkyl)₀₋₁-(substituted heterocyclyl);
—COOH;
—CO—O-alkyl;
—CO-alkyl;
—S(O)₀₋₂-alkyl;
—S(O)₀₋₂-(alkyl)₀₋₁aryl;
—S(O)₀₋₂-(alkyl)₀₋₁-(substituted aryl);
—S(O)₀₋₂-(alkyl)₀₋₁heteroaryl;
—S(O)₀₋₂-(alkyl)₀₋₁-(substituted heteroaryl);
—S(O)₀₋₂-(alkyl)₀₋₁heterocyclyl;
—S(O)₀₋₂-(alkyl)₀₋₁-(substituted heterocyclyl);
-(alkyl)₀₋₁-N(R₆₂₈)₂;
-(alkyl)₀₋₁-NR₆₂₈—CO—O-alkyl;
-(alkyl)₀₋₁-NR₆₂₈—CO-alkyl;
-(alkyl)₀₋₁-NR₆₂₈—CO-aryl;
-(alkyl)₀₋₁-NR₆₂₈—CO-(substituted aryl);
-(alkyl)₀₋₁-NR₆₂₈—CO-heteroaryl;
-(alkyl)₀₋₁-NR₆₂₈—CO-(substituted heteroaryl);
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;

R₂₂₈ is selected from:
-hydrogen;
-alkyl;
-alkenyl;
-aryl;
-substituted aryl;
-heteroaryl;
-substituted heteroaryl;
-alkyl-O-alkyl;
-alkyl-S-alkyl;
-alkyl-O-aryl;
-alkyl-S-aryl;
-alkyl-O-alkenyl;
-alkyl-S-alkenyl; and
-alkyl or alkenyl substituted by one or more substituents selected from:
   —OH;
   -halogen;
   —N(R₆₂₈)₂;
   —CO—N(R₆₂₈)₂;
   —CS—N(R₆₂₈)₂;
   —SO₂—N(R₆₂₈)₂;
   —NR₆₂₈—CO—C₁₋₁₀ alkyl;
   —NR₆₂₈—CS—C₁₋₁₀ alkyl;
   —NR₆₂₈—SO₂—C₁₋₁₀ alkyl;
   —CO—C₁₋₁₀ alkyl;
   —CO—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —CO-aryl;
   —CO-(substituted aryl);
   —CO-heteroaryl; and
   —CO-(substituted heteroaryl);

R₃₂₈ and R₄₂₈ are independently selected from hydrogen, alkyl, alkenyl, halogen, alkoxy, amino, alkylamino, dialkylamino, and alkylthio;

R₅₂₈ is H or C₁₋₁₀ alkyl, or R₅₂₈ can join with X to form a ring; or when R₁₂₈ is alkyl, R₅₂₈ and R₁₂₈ can join to form a ring;

each R₆₂₈ is independently H or C₁₋₁₀alkyl;

and pharmaceutically acceptable salts thereof.

In another embodiment, the IRM compound can be chosen from 1H-imidazo[4,5-c]pyridin-4-amines defined by Formula XXIX below:

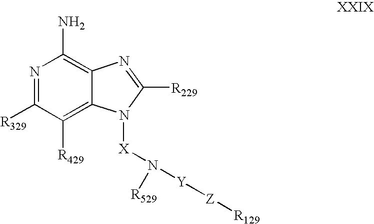

wherein

X is alkylene or alkenylene;

Y is —CO— or —CS;

Z is —NR₆₂₉—, —NR₆₂₉—CO—, —NR₆₂₉—SO₂—, or —NR₇₂₉—;

R₁₂₉ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkyl)₀₋₁-aryl;
—O-(alkyl)₀₋₁-(substituted aryl);
—O-(alkyl)₀₋₁-heteroaryl;
—O-(alkyl)₀₋₁-(substituted heteroaryl);
20-O-(alkyl)₀₋₁heterocyclyl;
   —O-(alkyl)₀₋₁-(substituted heterocyclyl);
   —COOH;
   —CO—O-alkyl;
   —CO-alkyl;
   —S(O)₀₋₂-alkyl;
   —S(O)₀₋₂-(alkyl)₀₋₁aryl;
   —S(O)₀₋₂₀-(alkyl)₀₋₁-(substituted aryl);
   —S(O)₀₋₂-(alkyl)₀₋₁heteroaryl;
   —S(O)₀₋₂-(alkyl)₀₋₁-(substituted heteroaryl);
30-S(O)₀₋₂-(alkyl)₀₋₁-heterocyclyl;
   —S(O)₀₋₂-(alkyl)₀₋₁-(substituted heterocyclyl);
   -(alkyl)₀₋₁-N(R₆₂₉)₂;
   -(alkyl)₀₋₁-NR₆₂₉—CO—O-alkyl;
   -(alkyl)₀₋₁-NR₆₂₉—CO-alkyl;
   -(alkyl)₀₋₁-NR₆₂₉—CO-aryl;
   -(alkyl)₀₋₁-NR₆₂₉—CO-(substituted aryl);
   -(alkyl)₀₋₁-NR₆₂₉—CO-heteroaryl;
   -(alkyl)₀₋₁-NR₆₂₉—CO-(substituted heteroaryl);
   —P(O)(O-alkyl)₂;
   —N₃;
   -halogen;
   -haloalkyl;
   -haloalkoxy;
   —CO-haloalkyl;
   —CO-haloalkoxy;
   —NO₂;
   —CN;
   —OH;
   —SH; and in the case of alkyl, alkenyl, and heterocyclyl, oxo;

R₂₂₉ is selected from:
-hydrogen;
-alkyl;
-alkenyl;
-aryl;
-substituted aryl;
-heteroaryl;
-substituted heteroaryl;
-alkyl-O-alkyl;
-alkyl-S-alkyl;
-alkyl-O-aryl;
-alkyl-S-aryl:
-alkyl-O-alkenyl;
-alkyl-S-alkenyl; and
-alkyl or alkenyl substituted by one or more substituents selected from:
   —OH;
   -halogen;
   —N(R₆₂₉)₂;
   —CO—N(R₆₂₉)₂;
   —CS—N(R₆₂₉)₂;
   —SO₂—N(R₆₂₉)₂;
   —NR₆₂₉—CO—C₁₋₁₀ alkyl;
   —NR₆₂₉—CS—C₁₋₁₀ alkyl;
   —NR₆₂₉—SO₂—C₁₋₁₀ alkyl;
   —CO—C₁₋₁₀ alkyl;
   —CO—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —CO-aryl;
   —CO-(substituted aryl);
   —CO-heteroaryl; and
   —CO-(substituted heteroaryl);

R₃₂₉ and R₄₂₉ are independently selected from hydrogen, alkyl, alkenyl, halogen, alkoxy, amino, alkylamino, dialkylamino, and alkylthio;

R₅₂₉ is H or C₁₋₁₀ alkyl, or R₅₂₉ can join with X to form a ring that contains one or two heteroatoms;

each R₆₂₉ is independently H or C₁₋₁₀alkyl;

R₇₂₉ is H or C₁₋₁₀ alkyl which may be interrupted by a heteroatom; or when R₁₂₉ is alkyl, R₇₂₉ and R₁₂₉ can join to form a ring;

and pharmaceutically acceptable salts thereof.
In another embodiment, the IRM compound can be chosen from 1-position ether or thioether substituted 1H-imidazo[4,5-c]pyridin-4-amines defined by Formula XXX below:

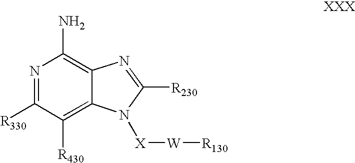

wherein:

X is —CH(R₅₃₀)—, —CH(R₅₃₀)-alkylene-, —CH(R₅₃₀)-alkenylene-,

or CH(R₅₃₀)-alkylene-Y-alkylene-;

Y is —O—, or —S(O)₀₋₂—;
—W—R₁₃₀ is selected from —O—R₁₃₀₋₁₋₅ and —S(O)₀₋₂—R₁₃₀₋₆;
R₁₃₀₋₁₋₅ is selected from
   —R₆₃₀—C(R₇₃₀)-Z-R₈₃₀-alkyl;
   —R₆₃₀—C(R₇₃₀)-Z-R₈₃₀-alkenyl;
   —R₆₃₀—C(R₇₃₀)-Z-R₈₃₀-aryl;
   —R₆₃₀—C(R₇₃₀)-Z-R₈₃₀-heteroaryl;
   —R₆₃₀—C(R₇₃₀)-Z-R₈₃₀-heterocyclyl;
   —R₆₃₀—C(R₇₃₀)-Z-H;
   —R₆₃₀—N(R₉₃₀)—C(R₇₃₀)—R₈₃₀-alkyl;
   —R₆₃₀—N(R₉₃₀)—C(R₇₃₀)—R₈₃₀-alkenyl;
   —R₆₃₀—N(R₉₃₀)—C(R₇₃₀)—R₈₃₀-aryl;
   —R₆₃₀—N(R₉₃₀)—C(R₇₃₀)—R₈₃₀-heteroaryl;
   —R₆₃₀—N(R₉₃₀)—C(R₇₃₀)—R₈₃₀-heterocyclyl;
   —R₆₃₀—N(R₉₃₀)—C(R₇₃₀)—R₁₀₃₀;
   —R₆₃₀—N(R₉₃₀)—SO₂—R₈₃₀-alkyl;
   —R₆₃₀—N(R₉₃₀)—SO₂—R₈₃₀-alkenyl;
   —R₆₃₀—N(R₉₃₀)—SO₂—R₈₃₀-aryl;
   R₆₃₀—N(R₉₃₀)—SO₂—R₈₃₀-heteroaryl;
   —R₆₃₀—N(R₉₃₀)—SO₂—R₈₃₀-heterocyclyl;
   —R₆₃₀—N(R₉₃₀)—SO₂—R₁₀₃₀;
   —R₆₃₀—N(R₉₃₀)—SO₂—N(R₅₃₀)—R₈₃₀-alkyl;
   —R₆₃₀—N(R₉₃₀)—SO₂—N(R₅₃₀)—R₈₃₀-alkenyl;
   —R₆₃₀—N(R₉₃₀)—SO₂—N(R₅₃₀)—R₈₃₀-aryl;
   —R₆₃₀—N(R₉₃₀)—SO₂—N(R₅₃₀)—R₈₃₀-heteroaryl;
   —R₆₃₀—N(R₉₃₀)—SO₂—N(R₅₃₀)—R₈₃₀-heterocyclyl;
   —R₆₃₀—N(R₉₃₀)—SO₂—NH₂;
   —R₆₃₀—N(R₉₃₀)—C(R₇₃₀)—N(R₅₃₀)-Q-R₈₃₀-alkyl;
   —R₆₃₀—N(R₉₃₀)—C(R₇₃₀)—N(R₅₃₀)-Q-R₈₃₀-alkenyl;
   —R₆₃₀—N(R₉₃₀)—C(R₇₃₀)—N(R₅₃₀)-Q-R₈₃₀-aryl;
   —R₆₃₀—N(R₉₃₀)—C(R₇₃₀)—N(R₅₃₀)-Q-R₈₃₀-heteroaryl;
   —R₆₃₀—N(R₉₃₀)—C(R₇₃₀)—N(R₅₃₀)-Q-R₈₃₀-heterocyclyl;
   —R₆₃₀—N(R₉₃₀)—C(R₇₃₀)—N(R₅₃₀)₂;

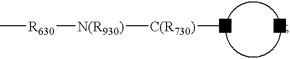

—R₆₃₀—N(R₉₃₀)—C(R₇₃₀)—N(R₁₁₃₀)-Q-R₈₃₀-alkyl;
—R₆₃₀—N(R₉₃₀)—C(R₇₃₀)—N(R₁₁₃₀)-Q-R₈₃₀-alkenyl;
—R₆₃₀—N(R₉₃₀)—C(R₇₃₀)—N(R₁₁₃₀)-Q-R₈₃₀-aryl;
—R₆₃₀—N(R₉₃₀)—C(R₇₃₀)—N(R₁₁₃₀)-Q-R₈₃₀-heteroaryl;
—R₆₃₀—N(R₉₃₀)—C(R₇₃₀)—N(R₁₃₀)-Q-R₈₃₀-heterocyclyl;
—R₆₃₀—N(R₉₃₀)—C(R₇₃₀)—N(R₁₁₃₀)H;
-alkenyl;
-aryl;
—R₆₃₀-aryl;
-heteroaryl;
-heterocyclyl;
—R₆₃₀-heteroaryl; and
—R₆₃₀-heterocyclyl;

Z is —N(R₅₃₀)—, —O—, or —S—;

Q is a bond, —CO—, or —SO₂—;

### A represents the atoms necessary to provide a 5- or 6-membered heterocyclic or heteroaromatic ring that contains up to three heteroatoms;

R₁₃₀₋₆ is selected from:
-alkyl;
-aryl;
-heteroaryl;
-heterocyclyl;
-alkenyl;
—R₆₃₀-aryl;
—R₆₃₀=heteroaryl; and
—R₆₃₀-heterocyclyl;

each R₅₃₀ is independently hydrogen, C₁₋₁₀ alkyl, or C₂₋₁₀ alkenyl;

R₆₃₀ is alkylene, alkenylene, or alkynylene, which may be interrupted by one or more —O— groups;

R₇₃₀ is ═O or ═S;

R₈₃₀ is a bond, alkylene, alkenylene, or alkynylene, which may be interrupted by one or more —O— groups;

R₉₃₀ is hydrogen, C₁₋₁₀ alkyl, or arylalkyl; or R₉₃₀ can join together with any carbon atom of R₆₃₀ to form a ring of the formula

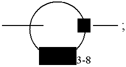

R₁₀₃₀ is hydrogen or C₁₋₁₀ alkyl; or R₉₃₀ and R₁₀₃₀ can join together to form a ring selected from

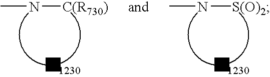

R₁₁₃₀ is C₁₋₁₀ alkyl; or R₉₃₀ and R₁₁₃₀ can join together to form a ring having the structure

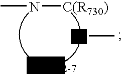

R₁₂₃₀ is C₂₋₇ alkylene which is straight chain or branched, wherein the branching does not prevent formation of the ring; and

R₂₃₀, R₃₃₀ and R₄₃₀ are independently selected from hydrogen and non-interfering substitutents;

and pharmaceutically acceptable salts thereof.

Illustrative non-interfering R₂₃₀ substituents include:
-alkyl;
-alkenyl;
-aryl;
-heteroaryl;
-heterocyclyl;
-alkylene-Y-alkyl;
-alkylene-Y-alkenyl;
-alkylene-Y-aryl; and
-alkyl or alkenyl substituted by one or more substituents selected from the group consisting of:
   —OH;
   -halogen;
   —N(R₅₃₀)₂;
   —C(O)—C₁₋₁₀ alkyl;
   —C(O)—O—C₁₋₁₀ alkyl;
   —N₃;
   -aryl;
   -heteroaryl;
   -heterocyclyl;
   —C(O)-aryl; and
   —C(O)-heteroaryl.

Illustrative non-interfering R₃₃₀ and R₄₃₀ substitutents include:

C₁₋₁₀ alkyl, C₂₋₁₀ alkenyl, C₂₋₁₀ alkynyl, C₁₋₁₀ alkoxy, C₁₋₁₀ alkylthio, amino, alkylamino, dialkylamino, halogen, and nitro.

In another embodiment, the IRM compound can be chosen from 1H-imidazo dimers of the formula (XXXI):

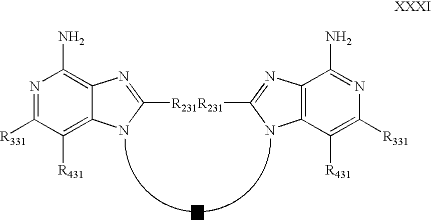

wherein:

A is a divalent linking group selected from the group consisting of:
straight or branched chain C₄₋₂₀ alkylene;
straight or branched chain C₄₋₂₀ alkenylene;
straight or branched chain C₄₋₂₀ alkynylene; and
-Z-Y—W—Y-Z-;

each Z is independently selected from the group consisting of:
straight or branched chain C₂₋₂₀ alkylene;
straight or branched chain C₄₋₂₀ alkenylene; and
straight or branched chain C₄₋₂₀ alkynylene;
any of which may be optionally interrupted by —O—, —N(R₅₃₁)—, or
—S(O)₂—;

each Y is independently selected from the group consisting of:
a bond;
—N(R₅₃₁)C(O)—;
—C(O)N(R₅₃₁)—;
—N(R₅₃₁)C(O)N(R₅₃₁)—;
N(R₅₃₁)S(O)₂—;
—S(O)₂N(R₅₃₁)—;
—OC(O)O—;
—OC(O)—;
—C(O)O—;
—N(R₅₃₁)C(O)O—; and
—OC(O)N(R₅₃₁)—;

W is selected from the group consisting of:
straight or branched chain C₂₋₂₀ alkylene;
straight or branched chain C₂₋₂₀ alkenylene;
straight or branched chain C₄₋₂₀ alkynylene;
straight or branched chain perfluoro C₂₋₂₀ alkylene;
C₁₋₄ alkylene-O—C₁₋₄ alkylene;
—C(O)—;
—S(O)₂—;
—OC(O)O—;
—N(R₅₃₁)C(O)N(R₅₃₁)—;

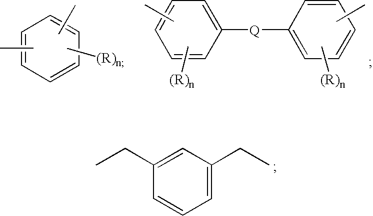

1,5-naphthylene;
2,6-pyridinylene;
1,2-cyclohexylene;
1,3-cyclohexylene;
1,4-cyclohexylene;
trans-1,4-cyclohexylene;

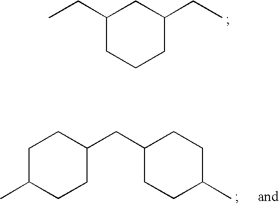

trans-5-norbornen-2,3-diyl;

wherein n is 0-4; each R is independently selected from the group consisting of C₁₋₄ alkyl, C₁₋₄ alkoxy, and halogen; and Q is selected from the group consisting of a bond, —CH₂—, and —O—;

R₂₃₁ is selected from the group consisting of:
-hydrogen;
-alkyl;
-alkenyl;
-aryl;
-substituted aryl;
-heteroaryl;
-substituted heteroaryl;
-alkyl-X-alkyl;
-alkyl-X-aryl;
-alkyl-X-alkenyl; and
-alkyl or alkenyl substituted by one or more substituents selected from the group consisting of:
   —OH;
   -halogen;
   —N(R₆₃₁)₂;
   —C(O)—N(R₆₃₁)₂;
   —C(S)—N(R₆₃ 1)₂;
   —S(O)₂—N(R₆₃₁)₂;
   —N(R₆₃₁)—C(O)—C₁₋₁₀ alkyl;
   —N(R₆₃₁)—C(S)—C₁₋₁₀ alkyl;
   —N(R₆₃₁)—S(O)₂—C₁₋₁₀ alkyl;
   —C(O)—C₁₋₁₀ alkyl;
   —C(O)—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —C(O)-aryl;
   —C(O)-(substituted aryl);
   —C(O)-heteroaryl; and
   —C(O)-(substituted heteroaryl);

R₃₃₁ and R₄₃, are each independently selected from the group consisting of:
-hydrogen;
-halogen;
-alkyl;
-alkenyl;
—X-alkyl; and
—N(R₆₃₁)₂;
or when taken together, R₃₃₁ and R₄₃₁ form a fused aryl or heteroaryl ring that is unsubstituted or substituted by one or more substituents selected from the group consisting of:
   -halogen;
   -alkyl;
   -alkenyl;
   —X-alkyl; and
   —N(R₆₃₁)₂;
or when taken together, R₃₃₁ and R₄₃₁ form a fused 5 to 7 membered saturated ring, containing 0 to 2 heteroatoms and unsubstituted or substituted by one or more substituents selected from the group consisting of:
   -halogen;
   -alkyl;
   -alkenyl;
   —X-alkyl; and
   —N(R₆₃₁)₂;

each R₅₃₁ is independently selected from the group consisting of:
hydrogen;
C₁₋₆ alkyl;
C₃₋₇ cycloalkyl; and
benzyl; or

when Y is —N(R₅₃₁)C(O)—, —C(O)N(R₅₃₁)—, —N(R₅₃₁)C(O)N(R₅₃₁)—, —N(R₅₃₁)S(O)₂—, —S(O₂)N(R₅₃₁)—, —N(R₅₃₁)C(O)O—, or —OC(O)N(R₅₃₁)— and the nitrogen of the N(R₅₃₁) group is bonded to Z, then R₅₃₁ can join with Z to form a ring having the structure

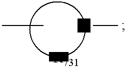

each R₆₃₁ is independently hydrogen or C₁₋₁₀ alkyl;

R₇₃₁ is C₃₋₈ alkylene; and

X is —O— or —S—;

with the proviso that if W is —C(O)—, —S(O)₂—, —OC(O)O—, or —N(R₅₃₁)C(O)N(R₅₃₁)— then each Y is a bond;
and pharmaceutically acceptable salts thereof.

In another embodiment, the IRM compound can be chosen from 6-, 7-, 8-, or 9-position aryl or heteroaryl substituted 1H-imidazo[4,5-c]quinolin-4-amines of the following Formula (XXXII):

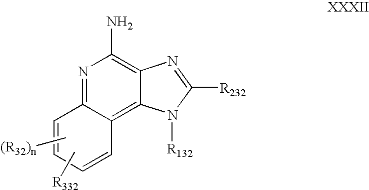

wherein:

R₃₂ is selected from the group consisting of alkyl, alkoxy, hydroxy, and trifluoromethyl;

n is 0 or 1;

R₁₃₂ and R₂₃₂ are independently selected from the group consisting of hydrogen and non-interfering substitutents;

R₃₃₂ is selected from the group consisting of:
-Z-Ar,
-Z-Ar′-Y—R₄₃₂,
-Z-Ar′-X—Y—R₄₃₂,
-Z-Ar′-R₅₃₂, and
-Z-Ar′-X—R₅₃₂;

Ar is selected from the group consisting of aryl and heteroaryl both of which can be unsubstituted or can be substituted by one or more substituents independently selected from the group consisting of alkyl, alkenyl, alkoxy, methylenedioxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—S(O)₀₋₂—,
—S(O)₂—N(R₈₃₂)—,
—C(R₆₃₂)—,
—C(R₆₃₂)—O—,
—O—C(R₆₃₂)—,
—O—C(O)—O—,
—N(R₈₃₂)-Q-,
—C(R₆₃₂)—N(R₈₃₂)—,
—O—C(R₆₃₂)—N(R₈₃₂)—,
—C(R₆₃₂)—N(OR₉₃₂)—,

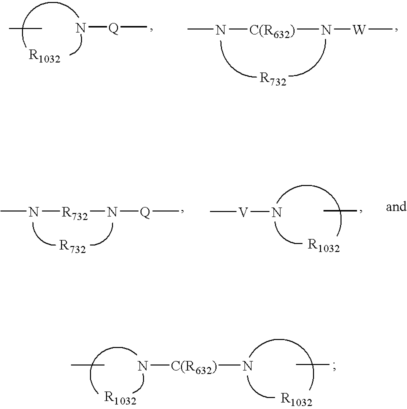

Z is selected from the group consisting of a bond, alkylene, alkenylene, and alkynylene;

R₄₃₂ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl,
oxo;

R₅₃₂ is selected from the group consisting of:

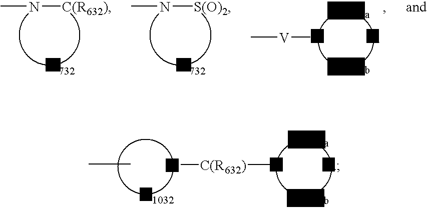

each R₆₃₂ is independently selected from the group consisting of ═O and ═S;

each R₇₃₂ is independently C₂₋₇ alkylene;

each R₈₃₂ is independently selected from the group consisting of hydrogen, alkyl, alkoxyalkylenyl, and arylalkylenyl;

R₉₃₂ is selected from the group consisting of hydrogen and alkyl;

each R₁₀₃₂ is independently C₃₋₈ alkylene;

A is selected from the group consisting of —O—, —C(O)—, —S(O)₀₋₂—, —CH₂—, and —N(R₄₃₂)—;

Q is selected from the group consisting of a bond, —C(R₆₃₂)—, —C(R₆₃₂)—C(R₆₃₂), —S(O)₂—, —C(R₆₃₂)—N(R₈₃₂)—W—, —S(O)₂—N(R₈₃₂)—, —C(R₆₃₂)—O—, and —C(R₆₃₂)—N(OR₉₃₂)—;

V is selected from the group consisting of —C(R₆₃₂)—, —O—C(R₆₃₂)—, —N(R₈₃₂)—C(R₆₃₂)—, and —S(O)₂—;

W is selected from the group consisting of a bond, —C(O)—, and —S(O)₂—; and

a and b are independently integers from 1 to 6 with the proviso that a+b is <7;

and pharmaceutically acceptable salts thereof.

Illustrative non-interfering R₁₃₂ substituents include:
—R₄₃₂,
—X—R₄₃₂,
—X—Y—R₄₃₂,
—X—Y—X—Y—R₄₃₂, and
—X—R₅₃₂;

wherein:

each X is independently selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O-groups;

each Y is independently selected from the group consisting of:
—S(O)₀₋₂—,
—S(O)₂—N(R₈₃₂)—,
—C(R₆₃₂)—,
—C(R₆₃₂)—O—,
—O—C(R₆₃₂)—,
—O—C(O)—O—,
—N(R₈₃₂)-Q-,
—C(R₆₃₂)—N(R₈₃₂)—,
O—C(R₆₃₂)—N(R₈₃₂)—,
—C(R₆₃₂)—N(OR₉₃₂)—,

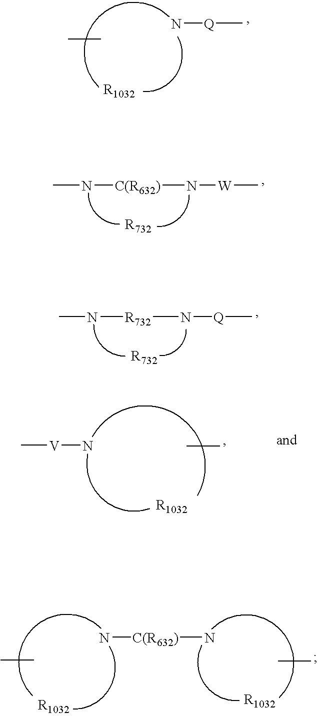

R₄₃₂ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅₃₂ is selected from the group consisting of:

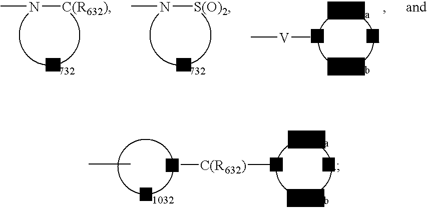

each R₆₃₂ is independently selected from the group consisting of ═O and ═S;

each R₇₃₂ is independently C₂₋₇ alkylene;

each R₈₃₂ is independently selected from the group consisting of hydrogen, alkyl, alkoxyalkylenyl, and arylalkylenyl;

each R₉₃₂ is independently selected from the group consisting of hydrogen and alkyl;

each R₁₀₃₂ is independently C₃₋₈ alkylene;

A is selected from the group consisting of —O—, —C(O)—, —S(O)₂—, —CH₂—, and —N(R₄₃₂)—;

each Q is independently selected from the group consisting of a bond, —C(R₆₃₂)—, —C(R₆₃₂)—C(R₆₃₂)—, —S(O)₂—, —C(R₆₃₂)—N(R₈₃₂)—W—, —S(O)₂—N(R₈₃₂)—, —C(R₆₃₂)—O—, and —C(R₆₃₂)—N(OR₉₃₂)—;

each V is independently selected from the group consisting of —C(R₆₃₂)—, —O—C(R₆₃₂)—, —N(R₈₃₂)—C(R₆₃₂)—, and —S(O)₂—;

each W is independently selected from the group consisting of a bond, —C(O)—, and —S(O)₂—; and

a and b are independently integers from 1 to 6 with the proviso that a+b is <7;

Illustrative non-interfering R₂₃₂ substitutents include:
—R₄₃₂,
—X—R₄₃₂,
—X—Y—R₄₃₂, and
—X—R₅₃₂;

wherein:

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—S(O)₀₋₂—,
—S(O)₂—N(R₈₃₂)—,
—C(R₆₃₂)—,
—C(R₆₃₂)—O—,
—O—C(R₆₃₂)—,
—O—C(O)—O—,
—N(R₈₃₂)-Q-,
—C(R₆₃₂)—N(R₈₃₂)—,
—O—C(R₆₃₂)—N(R₈₃₂)—,
—C(R₆₃₂)—N(OR₉₃₂)—,

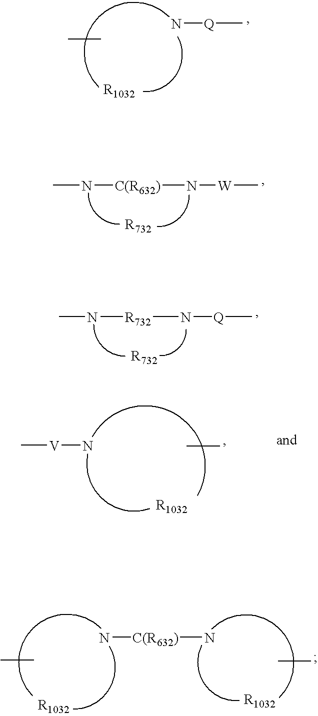

R₄₃₂ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅₃₂ is selected from the group consisting of:

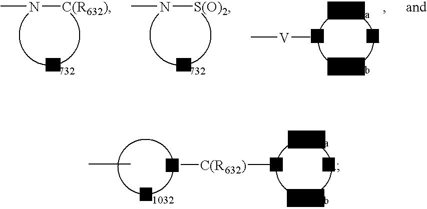

each R₆₃₂ is independently selected from the group consisting of ═O and ═S;

each R₇₃₂ is independently C₂₋₇ alkylene;

each R₈₃₂ is independently selected from the group consisting of hydrogen, alkyl, alkoxyalkylenyl, and arylalkylenyl;

R₉₃₂ is selected from the group consisting of hydrogen and alkyl;

each R₁₀₃₂ is independently C₃₋₈ alkylene;

A is selected from the group consisting of —O—, —C(O)—, —S(O)₀₋₂—, —CH₂—, and —N(R₄₃₂)—;

Q is selected from the group consisting of a bond, —C(R₆₃₂)—, —C(R₆₃₂)—C(R₆₃₂)—, —S(O)₂—, —C(R₆₃₂)—N(R₈₃₂)—W—, —S(O)₂—N(R₈₃₂)—, —C(R₆₃₂)—O—, and —C(R₆₃₂)—N(OR₉₃₂)—;

V is selected from the group consisting of —C(R₆₃₂)—, —O—C(R₆₃₂)—, —N(R₈₃₂)—C(R₆₃₂)—, and —S(O)₂—;

W is selected from the group consisting of a bond, —C(O)—, and —S(O)₂—; and

a and b are independently integers from 1 to 6 with the proviso that a+b is <7;

Herein, “non-interfering” means that the ability of the compound or salt to modulate (e.g., induce or inhibit) the biosynthesis of one or more cytokines is not destroyed by the non-interfering substituent.

As used herein, the terms “alkyl”, “alkenyl”, “alkynyl” and the prefix “alk-” are inclusive of both straight chain and branched chain groups and of cyclic groups, i.e. cycloalkyl and cycloalkenyl. Unless otherwise specified, these groups contain from 1 to 20 carbon atoms, with alkenyl and alkynyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene”, “alkenylene”, and “alkynylene” are the divalent forms of the “alkyl”, “alkenyl”, and “alkynyl” groups defined above. Likewise, “alkylenyl”, “alkenylenyl”, and “alkynylenyl” are the divalent forms of the “alkyl”, “alkenyl”, and “alkynyl” groups defined above. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. Similarly, the term “fluoroalkyl” is inclusive of groups that are substituted by one or more fluorine atoms, including perfluorinated groups (e.g., trifluoromethyl).

The term “aryl” as used herein includes carbocyclic aromatic rings or ring systems. Examples of aryl groups include phenyl, naphthyl, biphenyl, fluorenyl and indenyl.

The term “heteroatom” refers to the atoms O, S, or N.

The term “heteroaryl” includes aromatic rings or ring systems that contain at least one ring heteroatom (e.g., O, S, N). Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. Exemplary heterocyclic groups include pyrrolidinyl, tetrahydrofuranyl, morpholinyl, thiomorpholinyl, piperidinyl, piperazinyl, thiazolidinyl, imidazolidinyl, isothiazolidinyl, tetraliydropyranyl, quinuclidinyl, homopiperidinyl, homopiperazinyl, and the like.

The terms “arylene,” “heteroarylene,” and “heterocyclylene” are the divalent forms of the “aryl,” “heteroaryl,” and “heterocyclyl” groups defined above. Likewise, “arylenyl,” “heteroarylenyl,” and “heterocyclylenyl” are the divalent forms of the “aryl,” “heteroaryl,” and “heterocyclyl” groups defined above. For example, an alkylarylenyl group comprises an arylene moiety to which an alkyl group is attached.

Unless otherwise specified, the aryl, heteroaryl, and heterocyclyl groups of Formulas IX-XXXI can be unsubstituted or substituted by one or more substituents independently selected from the group consisting of alkyl, alkoxy, methylenedioxy, ethylenedioxy, alkylthio, haloalkyl, haloalkoxy, haloalkylthio, halogen, nitro, hydroxy, mercapto, cyano, carboxy, formyl, aryl, aryloxy, arylthio, arylalkoxy, arylalkylthio, heteroaryl, heteroaryloxy, heteroarylthio, heteroarylalkoxy, heteroarylalkylthio, amino, alkylamino, dialkylamino, heterocyclyl, heterocycloalkyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, heterocyclylcarbonyl, aryloxycarbonyl, heteroaryloxycarbonyl, arylthiocarbonyl, heteroarylthiocarbonyl, alkanoyloxy, alkanoylthio, alkanoylamino, aroyloxy, aroylthio, aroylamino, alkylaminosulfonyl, alkylsulfonyl, arylsulfonyl, heteroarylsulfonyl, aryldiazinyl, alkylsulfonylamino, arylsulfonylamino, arylalkylsulfonylamino, alkylcarbonylamino, alkenylcarbonylamino, arylcarbonylamino, arylalkylcarbonylamino, heteroarylcarbonylamino, heteroarylalkycarbonylamino, alkylsulfonylamino, alkenylsulfonylamino, arylsulfonylamino, arylalkylsulfonylamino, heteroarylsulfonylamino, heteroarylalkylsulfonylamino, alkylaminocarbonyl, dialkylaminocarbonyl, arylaminocarbonyl, arylalkylaminocarbonyl, alkenylaminocarbonyl, heteroarylaminocarbonyl, heteroarylalkylaminocarbonyl, alkylaminocarbonylamino, alkenylaminocarbonylamino, arylaminocarbonylamino, arylalkylaminocarbonylamino, heteroarylaminocarbonylamino, heteroarylalkylaminocarbonylamino and, in the case of heterocyclyl, oxo. If any other groups are identified as being “substituted” or “optionally substituted”, then those groups can also be substituted by one or more of the above enumerated substituents.

When a group (or substituent or variable) is present more that once in any Formula described herein, each group (or substituent or variable) is independently selected, whether explicitly stated or not. For example, for the formula —N(R₆₃₁)₂ each R₆₃₁ group is independently selected. In another example, when an R₂₃₂ and an R₃₃₂ group both contain an R₄₃₂ group, each R₄₃₂ group is independently selected. In a further example, when more than one Y group is present (i.e., R₂₃₂ and R₃₃₂ both contain a Y group) and each Y group contains one or more R₈₃₂ groups, then each Y group is independently selected, and each R₈₃₂ group is independently selected.

In certain embodiments, the immune response modifier is selected from the group consisting of imidazoquinoline amines, tetrahydroimidazoquinoline amines, imidazopyridine amines, 6,7-fused cycloalkylimidazopyridine amines, 1,2-bridged imidazoquinoline amines, imidazonaphthyridine amines, imidazotetrahydronaphthyridine amines, oxazoloquinoline amines, thiazoloquinoline amines, oxazolopyridine amines, thiazolopyridine amines, oxazolonaphthyridine amines, thiazolonaphthyridine amines, 1H-imidazo dimers fused to pyridine amines, quinoline amines, tetrahydroquinoline amines, naphthyridine amines, or tetrahydronaphthyridine amines, and combinations thereof.

### EXAMPLES

Objects and advantages of this invention are further illustrated by the following examples, but the particular materials and amounts thereof recited in these examples, as well as other conditions and details, should not be construed to unduly limit this invention. Unless otherwise provided, all percentages are given as w/w % (i.e., weight percents or wt-%).

TABLE 1

| Compound | Chemical Name | Reference |
|---|---|---|
| IRM1 | N-[4-(4-amino-2-ethyl-1H- | U.S. Pat. |
| | imidazo[4,5-c]quinolin- | No. 6,331,539^{#} |
| | 1-yl)butyl]methanesulfonamide | |
| IRM2 | N-{2-[4-amino-2-(ethoxymethyl)- | U.S. Pat. |
| | 1H-imidazo[4,5-c]quinolin-1-yl]-1,1- | No. 6,677,349 |
| | dimethylethyl}methanesulfonamide | Example 268 |

^{#}This compound is not specifically exemplified but can be readily prepared using the synthetic methods disclosed in the cited reference.

### Systemic Formulation

Formulations of IRM1 are prepared as described in Table 2 and Table 3 capable of being administered intra-venously or subcutaneously as follows:

TABLE 2
Intra-venous & Subcutaneous Formulations (% w/w)

| Ingredient | Placebo | 0.2 | 0.4 |
|---|---|---|---|
| IRM 1 | 0 | 0.2 | 0.4 |
| Citric Acid | 0.42 | 0.42 | 0.42 |
| Mannitol | 4.50 | 4.50 | 4.50 |
| Sodium Hydroxide, 1N, | ~4.00 | (3.80) | (3.80) |
| qs to pH = 5 | | | |
| Sterile Water for | ~91.38 | (90.88) | (90.88) |
| Injection, qs | | | |
| Total | 100 | 100 | 100 |

TABLE 3
Intra-venous & Subcutaneous
ormulations (% w/w)

| Ingredients | 0.15 | 0.15 |
|---|---|---|
| IRM 1 | 0.15 | 0.15 |
| Citric acid | 0.42 | — |
| Acetic Acid | — | 0.3 |
| Mannitol | 4.5 | 4.0 |
| 1N NaOH | 3.9 | 3.5 |
| Water | qs | qs |
| pH | 5.0 | 5.0 |

### Topical Formulation

IRM compounds are prepared as a 0.01, 0.3, 1.0, or 3% cream formulation as disclosed in US Patent Publication No. US 2003/0199538 and International Patent Publication No. WO 03/045391.

### Example 1

Following surgical excision of their lesions, patients with melanoma cutaneous metastasis or lentigo maligna melanoma lesions are treated at the excision site with IRM1 or IRM2 at a concentration of 0.01, 0.3, 1.0, or 3% cream formulation as disclosed in US Patent Publication No. US 2003/0199538 and International Patent Publication No. WO 03/045391. The placebo, IRM1, or IRM2 cream formulation is applied three times a week for four weeks.

Following the four weeks of topical application, patients are treated with an intra-venous (I.V.) formulation of IRM1 as described in Table 2 and Table 3. The I.V. formulation is injected three times a week for two to eight weeks with the placebo or a dosing level of 0.008, 0.016, or 0.032 mg/kg.

### Example 2

Following surgical excision of their lesions, patients with melanoma cutaneous metastasis or lentigo maligna melanoma lesions are treated with the intra-venous (I.V.) formulation as described above. The I.V. formulation is injected three times a week for two weeks with the placebo or a dosing level of 0.004 to 0.108 mg/kg.

Following the two weeks of systemic administration of IRM1, patients are treated topically with IRM1 or IRM2 at a concentration of 0.01, 0.3, 1.0, or 3% cream formulation as described above. The placebo, IRM1 or IRM2 cream formulations is applied three times a week for four weeks.

Once the four weeks of topically applied IRM1 or IRM2 is completed, patients resume systemic administration of IRM1 for an additional two to 24 weeks.

### Example 3

Patients with melanoma cutaneous metastasis or lentigo maligna melanoma lesions are treated at the lesion site with IRM1 or IRM2 at a concentration of 0.01, 0.3, 1.0, or 3% cream formulation as disclosed in US Patent Publication No. US 2003/0199538 and International Patent Publication No. WO 03/045391. The placebo, IRM1, or IRM2 cream formulation is applied three times a week for four weeks.

Following the four weeks of topical application, patients are treated with an intra-venous (I.V.) formulation of IRM1 as described in Table 2 or Table 3. The I.V. formulation is injected three times a week for two to eight weeks with the placebo or a dosing level of 0.004 to 0.108 mg/kg.

### Example 4

Patients with melanoma cutaneous metastasis or lentigo maligna melanoma lesions are treated with an intra-venous (I.V.) formulation as described above. The I.V. formulation is injected three times a week for two weeks with the placebo or a dosing level of 0.004 to 0.108 mg/kg.

Following the two weeks of systemic administration of IRM1, patients are treated topically with IRM1 or IRM2 at a concentration of 0.01, 0.3, 1.0, or 3% cream formulation as described above. The placebo, IRM1, or IRM2 cream formulations is applied three times a week for four weeks.

Once the four weeks of topically applied IRM1 or IRM2 is completed, patients resume systemic administration of IRM1 for an additional two to 24 weeks.

### Example 5

Topical formulations of IRM1 and a vehicle were formulated as shown in Table 3.

TABLE 3

| Materials | Vehicle | 5% IRM1 |
|---|---|---|
| IRM1 | — | 5.00 |
| Isostearic acid | 31.00 | 31.00 |
| Crodamol | 5.00 | 5.00 |
| Propylene glycol, USP | 9.00 | 9.00 |
| Methylparaben, NF | 0.20 | 0.20 |
| Ethylparaben, NF | 0.20 | 0.20 |
| Purified water, USP | 48.4 | 44.4 |
| Carbopol 980, NF | 0.90 | 0.60 |
| Poloxamer 188, NF | 3.75 | 3.75 |
| EDTA, USP | 0.05 | 0.05 |
| 20% w/w NaOH solution | 1.50 | 0.80 |
| (qs pH 5.8 +/− 0.2) | | |

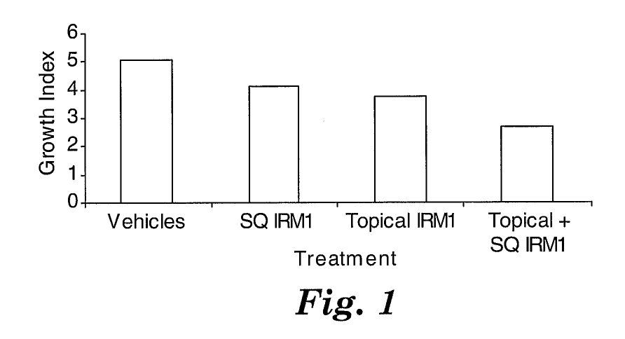

Eleven-week-old female Balb/c mice (Charles River Laboratories, Wilmington, Mass.) were injected intra-dermally with 5×10⁵ mouse colon carcinoma-26 cells (MC26) expressing luciferase (ATCC, Manassas, Va.) on day 0. Mice were divided into five groups: topical vehicle, subcutaneous vehicle (SQ vehicle), IRM1 topical (topical IRM1), IRM1 subcutaneous (SQ IRM1), and IRM1 topical and subcutaneous (topical+SQ IRM1). Eighteen hours after injection of the cells, 30 microliters of vehicle or 5% IRM1 topical formulations, described in Table 3, was applied to the tumor site. Six hours later, mice were injected subcutaneously with 10 millograms per kilogram of IRM1 in a 1 milligram per milliliter IRM1, 0.03M citrate buffered saline solution or a vehicle 0.03M citrate buffered saline solution. On days 1, 5, and 6 mice were anesthetized with vaporized 3% isoflurane and in vivo photon counts of luciferin were measured using a Xenogen IVIS imaging system (Alameda, Calif.) following the manufacturer's protocol. The tumor growth index for each treatment group was calculated by dividing each group's day 5 or day 6 tumor photon counts by their day 1 tumor photon counts. The results indicate a surprising benefit when a combination of topical and systemic routes were used. The results for the day 6 tumor growth are found in FIG. 1, in which the topical and SQ vehicles were averaged together (vehicles).

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A method of treating disease with immune response modifiers (IRMs) by administering at least one IRM compound via at least two different routes of delivery.

2. The method of claim 1, wherein there is only one IRM compound active moiety is used.

3. The method of claim 2, wherein two different salt forms of the IRM compound active moiety are used.

4. The method of claim 1, wherein at least two different IRM compound active moieties are used.

5. The method of claim 1, wherein the routes of delivery include local delivery and systemic delivery.

6. The method in claim 5, wherein the local route of delivery is topical delivery.

7. The method of claim 6, wherein topical delivery is achieved using an IRM-containing gel or cream formulation.

8. The method of claim 5, wherein systemic delivery is achieved by injection or oral delivery.

9. The method of claim 1, wherein the disease being treated is cancer.

10. The method of claim 9, wherein an IRM is delivered locally directly to the cancer and an IRM is delivered systemically to the entire body.

11. The method of claim 10, wherein the IRM delivered locally is injected directly into the cancer.

12. The method of claim 1, wherein the disease is a viral, fungal, protazoal, or bacterial infection.

13. A method of treating melanoma with an immune response modifier (IRM), the method comprising:
applying at least one IRM topically to a melanoma lesion on a subject in combination with separately administering at least one IRM to the subject systemically.

14. The method of claim 13, wherein the IRM administered topically is administered to a dermal or mucosal tissue.

15. The method of claim 14 wherein the IRM administered topically is administered to a vaginal, rectal, nasal, buccal, or pulmonary surface.

16. The method of claim 13 wherein the IRM is a compound having a 2-aminopyridine fused to a five membered nitrogen-containing heterocyclic ring.

17. The method of claim 16 wherein the immune response modifier is selected from the group consisting of imidazoquinoline amines, tetrahydroimidazoquinoline amines, imidazopyridine amines, 6,7-fused cycloalkylimidazopyridine amines, 1,2-bridged imidazoquinoline amines, imidazonaphthyridine amines, imidazotetrahydronaphthyridine amines, oxazoloquinoline amines, thiazoloquinoline amines, oxazolopyridine amines, thiazolopyridine amines, oxazolonaphthyridine amines, thiazolonaphthyridine amines, 1H-imidazo dimers fused to pyridine amines, quinoline amines, tetrahydroquinoline amines, naphthyridine amines, or tetrahydronaphthyridine amines, and combinations thereof.

18. The method of claim 16, wherein the immune response modifier is selected from the group consisting of imidazoquinoline amines, tetrahydroimidazoquinoline amines, imidazopyridine amines, and combinations thereof.

19. The method of claim 16, wherein the immune response modifier is selected from the group consisting of amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl or heteroaryl substituted imidazoquinoline amines, amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, thioether substituted imidazopyridine amines, and combinations thereof.

20. The method of claim 19, wherein the immune response modifier is selected from the group consisting of amide substituted imidazoquinoline amines, sulfonamide substituted imidazoquinoline amines, urea substituted imidazoquinoline amines, thioether substituted imidazoquinoline amines, 7-aryl substituted imidazoquinoline amines, 7-heteroaryl substituted imidazoquinoline amines, sulfonamide substituted tetrahydroimidazoquinoline amines, and combinations thereof.

21. The method of claim 17, wherein the immune response modifier is an imidazoquinoline amine.

22. The method of claim 19, wherein the immune response modifier is a sulfonamide substituted imidazoquinoline amine.

23. The method of claim 16, wherein the immune response modifier is selected from the group consisting of N-[4-(4-amino-2-ethyl-1H-imidazo[4,5-c]quinolin-1-yl)butyl]methanesulfonamide, N-{2-[4-amino-2-(ethoxymethyl)-1H-imidazo[4,5-c]quinolin-1-yl)butyl]-1,1-dimethyleethyl}methanesulfonamide, pharmaceutically acceptable salts thereof, and combinations thereof.

24. The method of any preceding claim, wherein an IRM is administered systemically in a formulation comprising:
a pharmaceutically acceptable acid;
a tonicity adjuster;
sterile water; and
optionally a pH adjuster;
with the proviso that the IRM is other than 1-(2-methylpropyl)-1H-imidazo[4,5-c]quinolin-4-amine or 4-amino-α,α-dimethyl-1H-imidazo[4,5-c]quinoline-1-ethanol.

25. The method of claim 24, wherein the formulation comprises 0.4 wt-% to 0.5 wt-% citric acid, 4 wt-% to 5 wt-% mannitol, and water, wherein the formulation is adjusted to a pH of 5 with the pH adjuster.

26. The method of claim 24, wherein the formulation comprises 0.2 wt-% to 0.5 wt-% acetic acid, 4 wt-% to 5 wt-% mannitol, and water, wherein the formulation is adjusted to a pH of 5 with the pH adjuster.
